(19) United States
(12) Patent Application Publication
(10) Pub. No.: US 2007/0254953 A1
(43) Pub. Date: Nov. 1, 2007

(54) Topical compositions of urea and ammonium lactate

(75) Inventors: Moshe Arkin, Kfar-Shemaryahu (IL); Nir Avram, Meitar (IL); Olga Buriakovsky, Beer-Sheva (IL); Amira Zeevi, Omer (IL); Benjamin Schneider, Hod-HaSharon (IL); Stephen Cherkez, Caesarea (IL); Stella Nivy, Tel-Aviv (IL)

Correspondence Address: Martin D. Moynihan, PRTSI, Inc., P.O. Box 16446, Arlington, VA, 22215, US

(73) Assignee: Perrigo Israel Pharmaceuticals Ltd., Petach-Tikva (IL)

(21) Appl. No.: 11/716,656

(22) Filed: Mar. 12, 2007

Related U.S. Application Data
(63) Continuation of application No. 10/850,461, filed on May 21, 2004.
(60) Provisional application No. 60/494,579, filed on Aug. 13, 2003.
(60) Provisional application No. 60/494,581, filed on Aug. 13, 2003.
(60) Provisional application No. 60/510,554, filed on Oct. 14, 2003.
(60) Provisional application No. 60/527,279, filed on Dec. 8, 2003.

(51) Int. Cl. A61K 31/19; A61P 17/00

(52) U.S. Cl. 514/557

(57) ABSTRACT

Pharmaceutical, cosmetic and cosmeceutical compositions for topical application, containing, as active ingredients, urea and/or derivatives thereof and alpha-hydroxy acid and/or an ammonium salt thereof, such as ammonium lactate, processes of manufacturing same and use of same in the treatment of medical and cosmetic skin and scalp conditions.

### RELATED APPLICATIONS

This application is a continuation of U.S. patent application Ser. No. 10/850,461 filed May 21, 2004, which claims the benefit of U.S. Provisional Patent Applications Nos. 60/494,579 filed Aug. 13, 2003, 60/494,581 filed Aug. 13, 2003, 60/510,554 filed Oct. 14, 2003 and 60/527,279 filed Dec. 8, 2003, the contents of which are hereby incorporated by reference in their entirety.

### FIELD AND BACKGROUND OF THE INVENTION

The present invention relates to novel pharmaceutical, cosmetic and cosmeceutical compositions for topical application, and their use in the treatment of medical, cosmetic and cosmeceutical conditions such as dry skin and/or scalp.

Dry skin is a common condition associated with a plurality of disorders and frequently requires therapeutic intervention.

Dermatologists often call dry skin in later life “xerosis” or “ichthyosis”. Xerosis is a term used to describe abnormal skin dryness. Ichthyosis is a term used to described a group of cutaneous disorders characterized by increased or aberrant keratinisation, and resulting in non-inflammatory scaling of the skin. There are at least 20 varieties of ichthyosis, including inherited and acquired forms. Further details regarding xerosis and ichthyosis can be found in “Atlas of Clinical Dermatology” by Anthony du Vivier, 3rd edition (Jul. 17, 2002) Publisher: Churchill Livingstone, which is incorporated herein by reference.

Dry skin often leads to dermatitis, a condition in which the skin becomes red and itchy, and which is typically characterized by a crazy-paving appearance on the lower legs (eczema craquele) or round patches scattered over the trunk and limbs (a dry form of nummular dermatitis). In some cases of dermatitis, such as, for example, winter itch, 7th age itch, or senile pruritus, the dry skin is just itchy, without much of a rash.

Dry skin results from, or is aggravated by, low humidity, sunlight, abrasive clothing and/or a repeated use of soaps, detergents or other lipid solvents, and is further strongly influenced by factors such as age, race, genetics, climate and lifestyle.

Numerous humidifying topical preparations containing emollients and moisturizers have been used over the years in the treatment of dry skin and more acute dermatological disorders which exhibit dry skin symptoms, such as, for example, ichthyosis, psoriasis, actinic damage, eczema and the like.

As is known in the art, the terms “moisturizer” (to add moisture) and “emollient” (to soften) are interchangeable as they describe different effects of the same agents on the skin, as is further detailed hereinunder.

“Moisturizers” is a general term used to describe substances that exert two basic actions: humectants, which are introduced into the stratum corneum to increase its water holding capacity; and occlusives, which provide a layer of oil on the surface of the skin to slow water loss and thus increase the moisture content of the stratum corneum. Some moisturizers contain both occlusives and humectants.

“Emollients” is a general term used to describe substances that cover the surface of the stratum corneum so as to prevent moisture loss, thus resulting in the closure of microcracks and fissures and restoration of the natural epidermal barrier. (Marie Loden, Clinics in Dermatology, 21, 145-157, 2003).

Herein, the terms “moisturizer”, “humectant”, “emollient” and the term “hydrating agent” are used interchangeably.

Ammonium lactate is a well-known emollient. Presently available commercial preparations that contain ammonium lactate include, for example, creams or lotions which comprise up to 12% ammonium lactate (e.g., Lac-Hydrin 12% and Lac-Hydrin 5%, marketed by Westwood Squibb, a division of Bristol-Myers Squibb). However, these commercial preparations are disadvantageous as they are characterized by low absorption and high stickiness.

Urea is a commonly used humectant. Urea is used in various biological systems, serving, inter alia, as a modifier of protein solubility. Urea is known to exert antibacterial activity as well as protein complexes denaturation activity. In topical applications, urea in known to act as a penetrating moisturizer with high osmotic activity, attributed to its capability to break hydrogen bonds in the outer layers of the stratum corneum, thus dispersing epidermal keratin and exposing water-binding sites. Urea also has a stabilizing effect on the stratum corneum barrier, which can be demonstrated by reduction of trans-epidermal water loss (TEWL) and of irritative hyperemia produced by the application of an irritant (John Ademola et al., Am. J. Clin. Dermatol 3(3), 217-222, 2002).

Urea-containing preparations have recently been used to treat various afflictions related to dry skin, whereby preparations that contain urea concentration lower than 10 weight percentages have generally been used as skin moisturizers, while preparations that contain urea concentration of 10 weight percentages or higher have been used as skin remedies, treating sever cases of dry, rough skin, such as ichthyosis and psoriasis. A representative example of a family of 40% urea-containing preparations is the Carmol®40 cream, gel and lotion (marketed by Doak Dermatologics, a subsidiary of Bradley Pharmaceuticals Inc.), which is known as a tissue softener. Another commercially available preparation is U-Lactin, which comprises 10 weight percentages of urea and 2 weight percentages of lactic acid. Although lactic acid is a known hydrating agent, its concentration in this preparation is relatively low and therefore provides no additive hydrating effect.

A number of comparative studies relating to the therapeutic efficacy of urea and ammonium lactate in the context of xerosis have been conducted. For example, a 40% urea cream and a 12% ammonium lactate lotion were compared in a clinical study for their efficiency in the treatment of xerosis. At day 14 of this study, the 40% urea cream was found to be superior to the 12% ammonium lactate lotion by most of the instrumental and clinical assessments (John Ademola et al., Am. J. Clin. Dermatol 3(3), 217-222, 2002).

Nevertheless, topical formulations containing high concentrations of urea suffer many disadvantages. For example, commercially available formulations such as the above-mentioned Carmol®40 have an alkaline pH, namely a pH value higher than 8.0. Such a pH value is much higher than that of the natural skin (about 5.5), and may therefore cause irritations when applied. Moreover, topical application of formulations that contain high urea concentrations are typically associated with an unpleasant odor of ammonia, formed by the decomposition of urea, stickiness, and white stains that remain on the skin and clothing after the evaporation of the solvent.

European Patent Application No. 0101887A2 discloses cosmetic compositions that comprise an aqueous solution of urea or derivatives thereof, in a concentration of between 0.5 M and 12 M, and an ammonium salt of an unreactive acid, which is added to adjust the pH of the solution to between 6.0 and 8.0. According to the teachings of this patent application, the ammonium salt is aimed at retarding the production of titratable alkali from the aqueous urea solution, to thereby prolong the shelf-life of the composition. Preferred ammonium salts, according to this patent application, include ammonium salts of strong acids such as carboxylic acids having up to four carbon atoms. The instability of urea in aqueous solutions is widely taught by this reference.

Hence, although both urea and ammonium lactate have been shown to act as highly efficient hydrating agents, and thus may serve as potent agents for treating dry skin conditions, the presently available topical formulations that comprise high concentrations of either urea or ammonium lactate suffer severe limitations.

While conceiving the present invention, it was envisioned that combining the benefits of urea and ammonium lactate in a stable topical compositions that contain relatively high concentrations of these ingredients would result in a highly potent composition for treating a variety of dermatological conditions, and particularly dry skin and scalp conditions and associated disorders.

Formulations that include urea and ammonium lactate have been described in the art.

For example, Gloor M. et al. (Skin Pharmacol Appl Skin Physiol 15, 35-43, 2002) have recently conducted a study investigating whether a combination of urea and ammonium lactate produced greater stratum corneum hydration than either component, in the same concentration, alone. The tested formulations included 5% ammonium lactate, 5% urea, 3% ammonium lactate in combination with 3% urea and 5% ammonium lactate in combination with 5% urea. According to the teachings of Gloor et al., the results obtained with the formulations combining urea and ammonium lactate were similar to those obtained with each component alone, at the tested concentrations. Furthermore, the obtained results showed that the effect of the 5% combined formulation was identical to the 3% combined formulation. Moreover, as this study was carried out with subjects that have healthy skin, it does not implicate the effect of these components, at the tested concentrations, in the treatment of dry skin and scalp conditions.

JP 59020217 (to Kawaken Fine Chemical KK) describes an aqueous, jelly-like composition containing between 1 and 48 weight percentages urea, an ammonium compound and a carboxyvinyl polymer. The pH of the composition is adjusted to 5.5 to 7.5, by adding a base made up of hydroxides of alkali metals, alkanolamines, basic amino acids and aqueous ammonia. According to the teachings of this patent, ammonium lactate can be one of the ammonium compounds added to the composition. However, further according to the teachings of this patent, the concentration of the ammonium compound should be relatively low, namely between 0.5 and 5 weight percentages, since at higher concentrations the resistance of the aqueous jelly-like composition to low temperatures is prohibitally reduced.

JP 59020217 further teaches that the combination of all the components present in the disclosed composition synergistically provides for inhibition of the decomposition of urea. It is therefore implied in this patent that the stability of the composition would be reduced if any of the constitutional components would be missing.

U.S. Pat. No. 5,679,324 (to Procter & Gamble Co.) discloses fragrance compositions, to which skin moisturizers such as, for example, urea or ammonium lactate can be added. The concentrations of the moisturizers in the composition are not disclosed. According to the teachings of this patent, urea, in a concentration of between 0.1% and about 10%, may further be added to the compositions as an optional medicament that may be included in the composition. U.S. Pat. No. 5,679,324 therefore teaches compositions that may optionally comprise urea or ammonium lactate and fails to teach compositions in which these substances are combined as active ingredients for the treatment of dry skin or scalp.

U.S. Pat. No. 6,086,903 (to Proctor & Gamble Company) discloses a personal treatment composition that comprises an enduring perfume composition. According to the teachings of this patent, urea or ammonium lactate may optionally be added to the composition as a moisturizer, in a concentrations of, as arbitrarily stated, between 0.1% and 20%, preferably, as stated, low concentration of between 2% and 5%. Again, this reference teaches compositions in which urea or ammonium lactate is an optional adjuvants, used in low concentrations.

U.S. Pat. No. 6,316,428 (to Crandall; Wilson Trafton) discloses a method of treating keratinous tissue of a human or animal, which is effected by topically applying to the keratinous tissue a composition comprising lecithin, poloxamer 407, a solvent and water. Urea and ammonium lactate, in relatively low concentrations of 2% and 5%, respectively, are taught by this patent as examples of molecules that may be delivered, separately, into the skin by the claimed composition.

U.S. Patent Application No. 20020151446 (to Playtex Products, Inc.) discloses a cleanser composition that comprises a mild surfactant system, a moisturizer system and a solvent system, wherein the mild surfactant system amounts to less than 17 weight percentages of the total weight of the composition, and the composition is delivered as a foam. The composition has a pH that ranges between about 4 and about 9. This patent application teaches that ammonium lactate can be one of the usable surfactants, in a concentration that ranges between about 0.1 weight percentages and about 15 weight percentages, and as one of the suitable moisturizers in an amount that ranges between about 0.1 weight percentages and about 6 weight percentages. Urea may also be included in the disclosed composition, in addition to ammonium lactate, as a humectant in an amount that ranges between about 1 weight percentages and about 5 weight percentages. Hence, the composition disclosed in this patent application comprises low concentrations of urea and ammonium lactate and is aimed at cleansing and conditioning of hair and skin, and not for the treatment of diseased or compromised skin.

All the compositions described above include urea and ammonium lactate in relatively low concentrations. The prior art therefore teaches either compositions that comprise high concentrations of urea or ammonium lactate, which, as is discussed hereinabove, suffer severe limitations and hence are disadvantageous, or compositions that comprise a combination of urea and ammonium lactate, whereby these substances are used in relatively low concentrations and therefore do not serve as efficient active ingredients for treating dry skin and scalp conditions.

There is thus a widely recognized need for, and it would be highly advantageous to have, topical compositions for treating dry skin and scalp conditions and related disorders, as well as other medical, cosmetic and cosmeceutical disorders, which include high and effective concentrations of urea and ammonium lactate and/or related substances and are devoid of the above limitations.

### SUMMARY OF THE INVENTION

The present inventors have now surprisingly found that topical compositions that comprise relatively high concentrations of urea and/or a derivative thereof and an alpha-hydroxy acid and/or a salt thereof, such as ammonium lactate, can serve as stable and efficient pharmaceutical, cosmetic and cosmeceutical compositions for the treatment of various dermatological disorders (e.g., dry skin and/or scalp).

Hence, according to one aspect of the present invention there is provided a pharmaceutical, cosmetic or cosmeceutical composition for topical application, which comprises urea and/or a derivative thereof, an alpha-hydroxy acid and/or a salt thereof and a pharmaceutically, cosmetically or cosmeceutically acceptable carrier, wherein the concentration of the urea and/or the derivative thereof is greater than 5 weight percentages of the composition and the concentration of the alpha-hydroxy acid and/or the salt thereof is greater than 5 weight percentages of the composition.

Preferably, the alpha-hydroxy acid is lactic acid, and more preferably, it is present in the composition of the present invention in the form of an ammonium salt thereof, namely, ammonium lactate.

Further preferably, the composition comprises urea.

Hence, according to another aspect of the present invention there is provided a pharmaceutical, cosmetic or cosmeceutical composition for topical application, which comprises urea, ammonium lactate and a pharmaceutically, cosmetically or cosmeceutically acceptable carrier, wherein the concentration of the urea is greater than 5 weight percentages of the composition and the concentration of the ammonium lactate is greater than 5 weight percentages of the composition.

According to further features in preferred embodiments of the invention described below, each of the pharmaceutical, cosmetic or cosmeceutical compositions of the present invention is packaged in a packaging material and identified in print, in or on the packaging material, for use in the treatment of a medical, cosmeceutic and/or cosmetic condition, such as, but not limited to, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, and xeroderma pigmentosum.

The total concentration of the urea and/or the derivative thereof and the alpha-hydroxy acid and/or the salt thereof (e.g., ammonium lactate) preferably ranges between about 11 weight percentages and about 60 weight percentages of the composition, more preferably between about 20 weight percentages and about 40 weight percentages of the composition, and most preferably it is about 32 weight percentages of the composition.

The concentration of the urea and/or the derivative thereof preferably ranges to between about 5.1 weight percentages and about 40 weight percentages of the composition, more preferably between about 15 weight percentages and about 25 weight percentages of the composition, and most preferably it is about 20 weight percentages of the composition.

The concentration of the alpha-hydroxy acid and/or the salt thereof (e.g., ammonium lactate) preferably ranges between 5.1 weight percentages and about 20 weight percentages of the composition, more preferably between about 8 weight percentages and about 16 weight percentages of the composition and even more preferably it is between about 10 weight percentages and about 16 weight percentages of the composition.

According to further features in preferred embodiments of the invention described below, each of the pharmaceutical, cosmetic or cosmeceutical compositions of the present invention can be in a form selected from the group consisting of a cream, an ointment, a paste, a gel, a lotion, a milk, a suspension, an aerosol, a spray, a foam, a shampoo, a hair conditioner, a serum, a swab, a pledget, a pad and a soap. The presently preferred forms are a foam, a cream and an ointment.

According to still further features in the described preferred embodiments each of the compositions of the present invention further comprises one or more additional active ingredients such as, but not limited to, an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

According to still further features in the described preferred embodiments each of the compositions of the present invention further comprises one or more ingredient(s) such as, but not limited to, a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant, a propellant and a surfactant. The one or more ingredient(s) preferably comprise allantoin and/or urazole.

The pharmaceutical, cosmetic or cosmeceutical compositions of the present invention preferably has a pH value that ranges between about 4 and about 7, more preferably between about 5 and about 6.

The pharmaceutical, cosmetic or cosmeceutical compositions of the present invention are preferably devoid of an enduring perfume composition.

According to yet another aspect of the present invention there are provided processes of preparing the pharmaceutical, cosmetic or cosmeceutical compositions of the present invention. Each of the processes comprises admixing urea and/or a derivative thereof, an alpha-hydroxy acid and/or a salt thereof (e.g., ammonium lactate) and a pharmaceutically, cosmetically or cosmeceutically acceptable carrier, such that the concentration of the urea and/or the derivative thereof is greater than 5 weight percentages of the composition and the concentration of the alpha-hydroxy acid and/or the salt thereof (e.g., ammonium lactate) is greater than 5 weight percentages of the composition, as is described hereinabove.

According to further features in preferred embodiments of the invention described below, in cases where the composition further comprises any of the active ingredients or other ingredients described hereinabove, the processes further comprise admixing the active ingredient(s) or any other ingredient(s) with the urea and/or the derivative thereof, the alpha-hydroxy acid and/or the salt thereof and the carrier.

According to still another aspect of the present invention there are provided methods of treating a medical, cosmetic and/or cosmeceutical condition such as, for example, a condition associated with dry skin and/or scalp. The methods comprise topically applying onto one or more biological surface(s) of a subject in need thereof, a pharmaceutically, cosmetically or cosmeceutically effective amount of any one of the compositions described hereinabove.

According to further features in preferred embodiments of the invention described below, the topically applying is performed between one and four times a day, preferably twice a day.

According to still further features in the described preferred embodiments the topically applying is for a time period that ranges between about 1 day and about 30 days, preferably about 14 days.

According to still further features in the described preferred embodiments the one or more biological surface(s) is selected from the group consisting of a lateral aspect of a forearm, a lateral aspect of a leg, an elbow, a palm, a foot, a backhand, a back and a scalp.

The present invention successfully addresses the shortcomings of the presently known configurations by providing stable and efficient compositions containing high concentrations of at least two hydrating agents (urea or a derivative thereof and an alpha-hydroxy acid and/or the salt thereof such as ammonium lactate), for use in the treatment of various medical, cosmetic and cosmeceutical conditions, particularly conditions associated with dry skin and/or scalp.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are comparative plots presenting the skin hydration at the forearms (FIG. 1a) and the lower legs (FIG. 1b), as measured by corneometry, upon treatment with a composition of the present invention (MCC) and a commercially available urea cream composition (UC);

FIGS. 2a-b are bar graphs presenting the investigators' evaluations of the degrees of skin roughness on the forearms of subjects treated with a composition of the present invention (MCC, FIG. 2a) and with a commercially available urea cream composition (UC, FIG. 2b), by time periods;

FIGS. 3a-d are bar graphs presenting the investigators' evaluations of the change in skin roughness on the forearms of subjects treated with a composition of the present invention (MCC) on day 14 (FIG. 3a) and on day 28 (FIG. 3b), and with a commercially available urea cream composition (UC) on day 14 (FIG. 3c) and on day 28 (FIG. 3d);

FIGS. 4a-b are bar graphs presenting the investigators' evaluations of the degrees of skin roughness on the lower legs of subjects treated with a composition of the present invention (MCC, FIG. 4a) and with a commercially available urea cream composition (UC, FIG. 4b), by time periods;

FIGS. 5a-d are bar graphs presenting the investigators' evaluations of the change in skin roughness on the lower legs of subjects treated with a composition of the present invention (MCC) on day 14 (FIG. 5a) and on day 28 (FIG. 5b), and with a commercially available urea cream composition (UC) on day 14 (FIG. 5c) and on day 28 (FIG. 5d);

FIGS. 6a-b are bar graphs presenting the investigators' evaluations of the degrees of skin dryness on the forearms of subjects treated with a composition of the present invention (MCC, FIG. 6a) and with a commercially available urea cream composition (UC, FIG. 6b), by time periods;

FIGS. 7a-d are bar graphs presenting the investigators' evaluations of the change in skin dryness on the forearms of subjects treated with a composition of the present invention (MCC) on day 14 (FIG. 7a) and on day 28 (FIG. 7b), and with a commercially available urea cream composition (UC) on day 14 (FIG. 7c) and on day 28 (FIG. 7d);

FIGS. 8a-b are bar graphs presenting the investigators' evaluations of the degrees of skin dryness on the lower legs of subjects treated with a composition of the present invention (MCC, FIG. 8a) and with a commercially available urea cream composition (UC, FIG. 8b), by time periods;

FIGS. 9a-d are bar graphs presenting the investigators' evaluations of the change in skin dryness on the lower legs of subjects treated with a composition of the present invention (MCC) on day 14 (FIG. 9a) and on day 28 (FIG. 9b), and with a commercially available urea cream composition (UC) on day 14 (FIG. 9c) and on day 28 (FIG. 9d);

FIGS. 10a-b demonstrate the distribution of the subjects evaluation of MCC and UC regarding skin texture at day 0 (FIG. 10a) and at day 14 (FIG. 10b) of the treatment;

FIG. 11 demonstrates the distribution of the subjects' evaluation of MCC and UC regarding in-package odor at day 0;

FIG. 12 demonstrates the distribution of the subjects evaluation of MCC and UC regarding odor quality at day 14; and

FIGS. 13a-b demonstrate the distribution of the subjects evaluation of MCC and UC regarding skin smell at day 0 (FIG. 13a) and at day 14 (FIG. 13b) of the treatment.

### DESCRIPTION OF THE PREFERRED EMBODIMENTS

The present invention is of compositions for topical application, which can be efficiently used in the treatment of various medical, cosmetic and cosmeceutical conditions. Specifically, the present invention is of (i) compositions for topical application, which contain, as active ingredients, urea and/or derivatives thereof and alpha-hydroxy acids and/or salts thereof (e.g., ammonium lactate); (ii) processes of preparing these compositions; and (iii) their use in treating medical, cosmetic and/or cosmeceutical skin and/or scalp conditions such as, but not limited to, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, and xeroderma pigmentosum.

The principles and operation of the compositions, processes and methods according to the present invention may be better understood with reference to the Examples and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As is discussed in detail hereinabove, urea and ammonium lactate are both known as efficient hydrating agents, which may therefore serve as potent agents for treating conditions such as dry skin and scalp. The hydrating efficacy and hence, the therapeutic or cosmetic performance of these substances highly depends on their concentration, requiring relatively high concentrations to achieve satisfactory therapeutic or cosmetic results. As is further discussed in detail in the Background section above, the presently available topical formulations that comprise high concentrations of either urea or ammonium lactate suffer from several major disadvantages, one being the instability of urea, which leads to its decomposition. The presently known formulations that comprise a combination of urea and ammonium lactate contain low concentrations of at least one of these substances and therefore cannot and do not serve as potent formulations for treating dermatological conditions such as dry skin or scalp.

In a search for a stable and efficient composition for treating medical and/or cosmetic conditions such as dry skin and scalp, which would overcome the disadvantages of the presently known formulations, the present inventors have surprisingly found that combining relatively high concentrations of urea and ammonium lactate in a formulation results in a stable composition which is highly efficient in the treatment of dry skin and/or scalp and is further characterized by improved absorption, after feel and comfort, as compared with the presently known formulations, and is devoid of unpleasant odors, stickiness and other adverse effects that accompany the use of the presently known formulations. The superior clinical efficacy and the improved and convenient application of a composition according to the present invention, as compared with presently available compositions, are demonstrated in the Examples section that follows.

Hence, according to one aspect of the present invention, there is provided a pharmaceutical, cosmetic or cosmeceutical composition for topical application, which comprises urea and ammonium lactate, as active ingredients, and a pharmaceutically, cosmetically or cosmeceutically acceptable carrier.

As used herein, the phrase “topical application” describes application onto a biological surface, e.g., skin or scalp. Hence, the phrase “a composition for topical application” describes a composition that is applied to a subject by direct laying or spreading on the skin, scalp or any other biological surface of the subject.

As used herein throughout the term “comprising” means that other steps and ingredients which do not affect the end results can be added. This term encompasses the terms “consisting of” and “consisting essentially of”.

The phrase “consisting essentially of” means that the composition may include additional ingredients, but only if the additional ingredients do not materially alter the basic and novel characteristics of the claimed compositions or methods.

The phrase “active ingredient” as used herein means an ingredient that exerts a pharmaceutical, cosmetic or cosmeceutical activity. As urea and ammonium lactate are known as hydrating agents, the phrase “active ingredient”, whenever used in the context of urea, ammonium lactate and related substances, refers to an ingredient that exerts a hydrating activity. As both urea and ammonium lactate serve as active ingredients in the composition of the present invention, the concentration of each of these substances is relatively high, so as to efficiently serve as hydrating agents.

Thus, the concentration of urea in the composition of the present invention is greater than 5 weight percentages of the composition, preferably greater than 6 weight percentages, more preferably greater than 7 weight percentages, more preferably greater than 8 weight percentages, more preferably greater than 9 weight percentages, more preferably greater than 10 weight percentages, more preferably greater than 11 weight percentages, more preferably greater than 12 weight percentages, more preferably greater than 13 weight percentages, more preferably greater than 14 weight percentages, more preferably greater than 15 weight percentages, more preferably greater than 16 weight percentages, more preferably greater than 17 weight percentages, more preferably greater than 18 weight percentages, more preferably greater than 19 weight percentages, and most preferably is about 20 weight percentages.

However, the concentration of urea in the composition of the present invention can further preferably be greater than 20 weight percentages and up to about 40 weight percentages.

The phrase “greater than” as used herein with respect to a numerical indication (e.g., a concentration) encompasses any number (integral or fractional) that is greater than the indicated number.

Hence, the urea concentration in the composition preferably ranges between, for example, about 5.1 weight percentages, and about 40 weight percentages, more preferably between about 15 and about 25 weight percentages, with about 20 weight percentages being the presently most preferred concentration.

The concentration of ammonium lactate in the composition of the present invention is preferably greater than 5 weight percentages of the composition, preferably greater than 6 weight percentages, more preferably greater than 7 weight percentages, more preferably greater than 8 weight percentages, more preferably greater than 9 weight percentages, more preferably greater than 10 weight percentages, more preferably greater than 11 weight percentages, and most preferably is about 12 weight percentages.

However, the concentration of ammonium lactate in the composition of the present invention can further preferably be greater than 12 weight percentages and up to 20 weight percentages or more. Hence, the ammonium lactate concentration in the composition preferably ranges between, for example, about 5.1 weight percentages and about 20 weight percentages, more preferably between about 8 and about 16 weight percentages, and more preferably between about 10 weight percentages and about 16 weight percentages. An ammonium lactate concentration that ranges between about 12 weight percentages and about 14 weight percentages is being the presently most preferred concentration.

As used herein throughout, the phrase “weight percentages” describes the weight percentages (of an ingredient) of the total weight of a composition containing same.

As used herein the term “about” refers to +10%.

The total concentration of the urea and ammonium lactate active ingredients in the composition of the present invention preferably ranges between about 11 and about 60 weight percentages, more preferably between about 20 and about 40 weight percentages of the composition, more preferably between about 28 and about 36 weight percentages, and most preferably it is about 32 weight percentages.

As has already been discussed hereinabove, one of the major limitations associated with compositions that comprise high concentrations of urea results from the instability of urea, which leads to its decomposition into ammonia and carbon dioxide. As is well known in the art, ammonia is characterized by strong unpleasant odor, and therefore its presence in a high concentration is highly undesirable in the context of topical compositions.

As the decomposition of urea occurs mainly under basic conditions, it may be avoided, to some extent, by adjusting the pH of the composition, so as to render the composition non-alkaline.

Nonetheless, the composition of the present invention was found to be stable, such that no decomposition products of urea were observed. Without being bound to any particular theory, it is assumed that the ammonium lactate, apart from being an active hydrating agent, serves as a pH adjusting agent and hence as a stabilizing agent that prevents the decomposition of urea.

However, in order to further strength the stability of the composition of the present invention, the composition is preferably formulated so as to have a pH value that ranges between about 4 and about 7, more preferably between about 5 and about 6. As the natural pH of the skin is 5.5, such a pH value is further preferred for the composition of the present invention, so as to avoid irritation.

While urea is a well known and widely used hydrating agent, some derivatives of urea are also known to exert hydration properties, as is described, for example, in EP Application No. 0101887 A2. Such urea derivatives can therefore be beneficially used as active ingredients in the composition of the present invention, in addition to or instead of urea.

Similarly, while ammonium lactate is a known hydrating agent, other hydrating agents that belong to the well-known alpha-hydroxy acids family, can be used as active ingredients in the compositions of the present invention. Such hydrating agents which are used for treating dry skin are described, for example, in U.S. Pat. Nos. 3,879,537, 3,920,835, 3,984,470, 3,988,470, 4,021,572, 4,105,783, 4,246,261, 4,363,815, 5,422,370, and 5,554,597.

Hence, according to another aspect of the present invention, there is provided a pharmaceutical, cosmetic or cosmeceutical composition for topical application, which comprises urea and/or a derivative thereof and an alpha-hydroxy acid and/or a salt thereof, as the active ingredients, and a pharmaceutically, cosmetically or cosmeceutically acceptable carrier.

The urea derivative according to the present invention preferably includes substituted urea, and can be generally described, for example, by the general formula:
R¹R²N—C(—O)—NR³R⁴

wherein each of R¹, R², R³ and R⁴ is independently selected from the group consisting of hydrogen, alkyl, cycloalkyl, alkenyl and aryl, or, alternatively, one of R¹ and R² and one of R³ and R⁴ are linked therebetween to thereby form a heteroalicyclic ring. As used herein, the term “alkyl” refers to a saturated aliphatic hydrocarbon including straight chain and branched chain groups. Preferably, the alkyl group has between 1 and 20 carbon atoms. Whenever a numerical range; e.g., “1-20”, is stated herein, it means that the group, in this case the alkyl group, may contain 1 carbon atom, 2 carbon atoms, 3 carbon atoms, etc., up to and including 20 carbon atoms. More preferably, it is a medium size alkyl having 1 to 10 carbon atoms. Most preferably, it is a lower alkyl having 1 to 4 carbon atoms. The alkyl group may be substituted or unsubstituted. When substituted, the substituent group can be, for example, hydroxy, halo, amino, nitro, cyano, alkoxy, aryloxy, thiohydroxy, thioalkoxy, thioaryloxy, sulfinyl, sulfonyl, sulfonamide, phosphonyl, phosphinyl, carbonyl, thiocarbonyl, thiocarboxy, C-amido, N-amido, C-carboxy, O-carboxy, and sulfonamido.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, hydroxy, halo, amino, nitro, cyano, alkoxy, aryloxy, thiohydroxy, thioalkoxy, thioaryloxy, sulfinayl, sulfonyl, sulfonamide, phosphonyl, phosphinyl, carbonyl, thiocarbonyl, thiocarboxy, C-amido, N-amido, C-carboxy, O-carboxy, and sulfonamido.

An “alkenyl” group refers to an alkyl group, as is defined hereinabove, which consists of at least two carbon atoms and at least one carbon-carbon double bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, hydroxy, halo, amino, nitro, cyano, alkoxy, aryloxy, thiohydroxy, thioalkoxy, thioaryloxy, sulfinyl, sulfonyl, sulfonamide, phosphonyl, phosphinyl, carbonyl, thiocarbonyl, thiocarboxy, C-amido, N-amido, C-carboxy, O-carboxy, and sulfonamido.

However, other derivatives of urea such as, for example, thiourea or dimeric forms of urea are also within the scope of the present invention.

The alpha-hydroxy carboxylic acid or the salt thereof according to the present invention can be generally described, for example, by the general formula:
RaRbC₁(OH)C₂(═O)OX
wherein:

X is hydrogen, alkyl, cycloalkyl, aryl, halide, or an ammonium ion, such that when X is an ammonium ion, O is negatively charged, or, alternatively, X is a C2-C10 alkyl that is attached to C₂; and

Ra and Rb are each independently hydrogen, halide, alkyl, alkenyl, alkynyl, cycloalkyl or aryl,

or a salt thereof.

As used herein, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it means that the group, in this case the alkyl group, may contain 1 carbon atom, 2 carbon atoms, 3 carbon atoms, etc., up to and including 20 carbon atoms. More preferably, it is a medium size alkyl having 1 to 10 carbon atoms. Most preferably, it is a lower alkyl having 1 to 4 carbon atoms. The alkyl group may be substituted or unsubstituted. When substituted, the substituent group can be, for example, hydroxy, halo, amino, nitro, cyano, alkoxy, aryloxy, thiohydroxy, thioalkoxy, thioaryloxy, heteroaryl, heteroalicyclic, sulfinyl, sulfonyl, sulfonamide, phosphonyl, phosphinyl, carbonyl, thiocarbonyl, thiocarboxy, C-amido, N-amido, C-carboxy, O-carboxy, and sulfonamido.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, hydroxy, halo, amino, nitro, cyano, alkoxy, aryloxy, thiohydroxy, thioalkoxy, thioaryloxy, heteroaryl, heteroalicyclic, sulfinyl, sulfonyl, sulfonamide, phosphonyl, phosphinyl, carbonyl, thiocarbonyl, thiocarboxy, C-amido, N-amido, C-carboxy, O-carboxy, and sulfonamido.

An “alkenyl” group refers to an alkyl group, as is defined hereinabove, which consists of at least two carbon atoms and at least one carbon-carbon double bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, hydroxy, halo, amino, nitro, cyano, alkoxy, aryloxy, thiohydroxy, thioalkoxy, thioaryloxy, heteroaryl, heteroalicyclic, sulfinyl, sulfonyl, sulfonamide, phosphonyl, phosphinyl, carbonyl, thiocarbonyl, thiocarboxy, C-amido, N-amido, C-carboxy, O-carboxy, and sulfonamido.

The term “heteroaryl” refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, cycloalkyl, aryl, heteroaryl, heteroalicyclic, hydroxy, alkoxy, aryloxy, thiohydroxy, thioalkoxy, thioaryloxy, cyano, halide, carbonyl, thiocarbonyl, nitro and/or amino.

The term “heteroalicyclic” refers to a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydro furane, tetrahydropyrane, morpholino and the like. The heteroalicyclic may be substituted or unsubstituted. When substituted, the substituent group can be, for example, alkyl, cycloalkyl, aryl, heteroaryl, heteroalicyclic, hydroxy, alkoxy, aryloxy, thiohydroxy, thioalkoxy, thioaryloxy, cyano, halide, carbonyl, thiocarbonyl, nitro and/or amino.

A “hydroxy” group refers to an —OH group.

An “alkoxy” group refers to both an —O-alkyl and an —O-cycloalkyl group, as defined herein.

An “aryloxy” group refers to both an —O-aryl and an —O-heteroaryl group, as defined herein.

A “thiohydroxy” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is hydrogen, alkyl, alkenyl, cycloalkyl, aryl, heteroaryl (bonded through a ring carbon) or heteroalicyclic (bonded through a ring carbon) as defined herein.

A “thiocarbonyl” group refers to a —C(═S)—R′ group, where R′ is as defined herein for R′.

A “C-carboxy” group refers to a —C(═O)—O—R′ groups, where R′ is as defined herein.

An “O-carboxy” group refers to an R′C(═O)—O— group, where R′ is as defined herein.

A “halo” or “halide” group refers to fluorine, chlorine, bromine or iodine.

A “trihalomethyl” group refers to a —CX₃ group wherein X is a halo group as defined herein.

A “sulfinyl” group refers to a —S(═O)—R′ group, where R′ is as defined herein.

A “sulfonyl” group refers to a —S(—O)₂—R′ group, where R′ is as defined herein.

A “S-sulfonamido” group refers to a —S(═O)₂—NR′R″ group, with R′ is as defined herein and R″ is as defined herein for R′.

A “N-sulfonamido” group refers to an R′S(═O)₂—NR″ group, where R′ and

R″ are as defined herein.

An “amino” or “amine” group refers to an —NR′R″ group where R′ and R″ are as defined herein.

A “C-amido” group refers to a —C(═O)—NR′R″ group, where R′ and R″ are as defined herein.

A “N-amido” group refers to an R′C(═O)—NR″ group, where R′ and R″ are as defined herein.

A “nitro” group refers to a —NO₂ group.

A “cyano” group refers to a —C≡N group.

The term “phosphonyl” describes an —O—P(═O)(OR′)(OR″) group, with R′ and R″ as defined hereinabove.

The term “phosphinyl” describes a —PR′R″ group, with R′ and R″ as defined hereinabove.

Thus, the alpha-hydroxy carboxylic acid according to the present invention can be in a form of the free acid, such that X in the formula above is hydrogen, or a salt thereof, or, alternatively, the alpha-hydroxy carboxylic acid can be in a form of an ester, such that X in the formula above is alkyl, cycloalkyl or aryl, as these terms are defined hereinabove, or a salt thereof in some cases where the alkyl, cycloalkyl or aryl is substituted. The alpha-hydroxy carboxylic acid can further be in a form of an acyl chloride, such that X in the formula above is halide, or in a form of a lactone, such that X is the formula above is an alkyl that is attached to C₂, or a salt thereof, in some cases where the alkyl is substituted.

Further alternatively and preferably, the alpha-hydroxy carboxylic acid can be in a form of an ammonium salt, such that X in the formula hereinabove is an ammonium ion. As an ammonium ion is a positively charged ion, O in the formula above is negatively charged in this case.

Representative examples of alpha-hydroxy carboxylic acid and salts thereof that are usable in the context of this aspect of the present invention include, without limitation, 2-hydroxyethanoic acid (glycolic acid); 2-hydroxypropanoic acid (lactic acid); 2-methyl 2-hydroxypropanoic acid (methyl lactic acid); 2-hydroxybutanoic acid; 2-hydroxypentanoic acid; 2-hydroxyhexanoic acid; 2-hydroxyheptanoic acid; 2-hydroxyoctanoic acid; 2-hydroxynonanoic acid; 2-hydroxydecanoic acid; 2-hydroxyundecanoic acid; 2-hydroxydodecanoic acid (alpha-hydroxy lauric acid); 2-hydroxytetradecanoic acid (alpha-hydroxy myristic acid); 2-hydroxyhexadecanoic acid (alpha-hydroxy palmitic acid); 2-hydroxyoctadecanoic acid (alpha-hydroxystearin acid); 2-hydroxyeicosanoic acid (alpha-hydroxy arachidonic acid); 2-phenyl 2-hydroxyethanoic acid (mandelic acid); 2,2-diphenyl 2-hydroxyethanoic acid (Benzylic acid); 3-phenyl 2-hydroxypropanoic acid (phenylacetic acid); 2-phenyl 2-methyl 2-hydroxyethanoic acid (atrolactic acid); 2-(4′-hydroxyphenyl) 2-hydroxyethanoic acid; 2-(4′-chlorophenyl 2-hydroxyethanoic acid; 2-(3′-hydroxy-4′-methoxyphenyl) 2-hydroxyethanoic acid; 2-(4′-hydroxy-3′-methoxyphenyl) 2-hydroxyethanoic acid; 3-(2-hydroxyphenyl) 2-hydroxypropanoic acid; 3-(4′-hydroxyphenyl) 2-hydroxypropanoic acid; 2-(3′,4′-dihydroxyphenyl) 2-hydroxyethanoic acid; and any salt thereof.

The concentration of urea and/or of its derivative is greater than 5 weight percentages of the composition, preferably greater than 6 weight percentages, more preferably greater than 7 weight percentages, more preferably greater than 8 weight percentages, more preferably greater than 9 weight percentages, more preferably greater than 10 weight percentages, more preferably greater than 11 weight percentages, more preferably greater than 12 weight percentages, more preferably greater than 13 weight percentages, more preferably greater than 14 weight percentages, more preferably greater than 15 weight percentages, more preferably greater than 160 weight percentages, more preferably greater than 17 weight percentages, more preferably greater than 18 weight percentages, more preferably greater than 19 weight percentages, and most preferably is about 20 weight percentages.

The concentration of urea and/or of its derivative in the composition of the present invention can further preferably be greater than 20 weight percentages and up to 40 weight percentages. Hence, the concentration of urea and/or of its derivative in the composition preferably ranges between 5.1 weight percentages and about 40 weight percentages, more preferably between about 15 and about 25 weight percentages, with about 20 weight percentages being the presently most preferred concentration.

The concentration of the alpha-hydroxy acid and/or of the salt thereof is greater than 5 weight percentages of the composition, preferably greater than 6 weight percentages, more preferably greater than 7 weight percentages, more preferably greater than 8 weight percentages, more preferably greater than 9 weight percentages, more preferably greater than 10 weight percentages, more preferably greater than 11 weight percentages, and most preferably is about 12 weight percentages.

The concentration of the alpha-hydroxy acid and/or of a salt thereof in the composition of the present invention can further preferably be greater than 12 weight percentages and up to 20 weight percentages. Hence, the concentration of the alpha-hydroxy acid and/or of the salt thereof in the composition preferably ranges between 5.1 weight percentages and about 20 weight percentages, more preferably between about 8 and about 16 weight percentages, whereby a concentration of between about 10 weight percentages and about 16 weight percentages being the presently most preferred concentration.

The total concentration of these two active ingredients preferably ranges between about 11 weight percentages and about 60 weight percentages, more preferably between about 20 and about 40 weight percentages and is preferably about 28-36 weight percentages, most preferably about 32 weight percentages.

Each of the compositions of the present invention, described hereinabove, further includes a pharmaceutically, cosmetically or cosmeceutically acceptable carrier.

As used herein, the term “pharmaceutically, cosmetically or cosmeceutically acceptable carrier” describes a carrier or a diluent that does not cause significant irritation to an organism and does not abrogate the biological activity and properties of the applied active ingredient(s).

Examples of acceptable carriers that are usable in the context of the present invention include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition.

Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions.

Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

By selecting the appropriate carrier and optionally other ingredients that can be included in the composition, as is detailed hereinbelow, the compositions of the present invention may be formulated into any pharmaceutical, cosmetic or cosmeceutical form normally employed for topical application. Hence, the compositions of the present invention can be, for example, in a form of a cream, an ointment, a paste, a gel, a lotion, a milk, a suspension, an aerosol, a spray, a foam, a shampoo, a hair conditioner, a serum, a swab, a pledget, a pad and a soap.

It will be appreciated that the final form of a topical composition plays an important role in its efficacy and its usage convenience.

The challenge in topically applying a composition is to achieve percutaneous penetration of the active ingredient to the site of treatment, in many cases the epidermis. At the same time, it is important that the composition should have desirable characteristics. Hence, application should be easy, smooth and should result in no irritation, discomfort or inconvenience. Desirably, the composition should not leave a residue on the surface of the skin. Topical compositions in forms such as gels, ointments, lotions, creams, pads and pastes are often very viscous, requiring substantial rubbing to achieve penetration of the active ingredient to the affected skin layer, an act which often results in discomfort and further irritation. Non-viscous creams and lotions require quick and dexterous application as they are inclined to flow off the site of treatment before penetration of the active ingredient is achieved.

Contrary to the above, foams are well suited for the topical application of compositions. Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system.

A foam composition has physical characteristics which are dependent, at least in part, upon the choice and relative amounts of components such as solvents, propellants and surfactants, which may be present in the composition. The combination of such components determines the stability of the foam, which may retain its foam-like structure upon application or be “a slow-breaking foam” or “a quick-breaking foam”, whereby this terminology relates to the behavior of the foam towards shearing action as is sustained when the foam is rubbed into or spread over a surface onto which it has been dispensed.

Many of the physical characteristics of foam compositions render it highly beneficial and advantageous over other forms. One such exemplary characteristic is the semi-solid to solid nature of the foam matrix, which allows the composition to be applied with the hand in any orientation without the risk of run off. Another beneficial characteristic of foams is their convenient application to large areas of the body surface. Furthermore, although foams can be water-based or hydroalcoholic, typically they are formulated with high alcohol content which, upon application to the skin of a user, quickly evaporates, driving the active ingredient through the upper skin layers to the site of treatment.

Hence, according to a preferred embodiment of the present invention, the compositions described herein are formulated in the form of a foam. More preferably, the compositions are in the form of a foam, which is formed by the passage of a pressurized mixture of a concentrate and a propellant through a nozzle. Preferably, the propellant is in the form of a compressed gas, typically a liquefiable gas. The mixture is preferably contained in a dispenser equipped with a dispensing head and valve, and pressurized with the propellant. Upon discharge of the composition through the dispensing head, the volatilization of the dispersed liquid droplets of propellant causes the dispensed concentrate to foam. Depending upon the precise formulation of the concentrate and the propellant, the dispensed product may range from a dense creamy foam to a light foam, dependent on desired aesthetics in the hand and when spread onto the substrate.

The concentration of the propellant in the composition preferably ranges between about 0.5 and about 60 weight percentages, more preferably between about 1 and about 20 weight percentages of the total composition.

Any propellant suitable for use in pharmaceutical, cosmetic or cosmeceutical compositions can be used herein. Non-limiting examples of suitable propellants include nitrous oxide, carbon dioxide, nitrogen, and hydrocarbon propellants such as propane, iso-butane, n-butane, isopentane, n-pentane, and dimethyl ether. Preferred propellants are selected from, for example, propane, iso-butane, n-butane, isopentane, n-pentane, and mixtures thereof. Chlorinated fluorocarbons such as 1,1-difluoro- or 1,1,1,2-tetrafluoroethane are also suitable but their use is being limited for environmental reasons. The propellants described above usually have a low boiling point and are in a gaseous form at room temperature in standard conditions.

According to another preferred embodiment of the present invention, the compositions described herein are formulated in the form of a cream or an ointment.

The compositions of the present invention can optionally further comprise a variety of components that are suitable for rendering the compositions more cosmetically or aesthetically acceptable or to provide the compositions with additional usage benefits. Such conventional optional components are well known to those skilled in the art and are referred to herein as “ingredients”. These include any cosmetically acceptable ingredients such as those found in the CTFA International Cosmetic Ingredient Dictionary and Handbook, 8th edition, edited by Wenninger and Canterbery, (The Cosmetic, Toiletry, and Fragrance Association, Inc., Washington, D.C., 2000). Some non-limiting representative examples of these ingredients include humectants, deodorants, antiperspirants, sun screening agents, sunless tanning agents, hair conditioning agents, pH adjusting agents, chelating agents, preservatives, emulsifiers, occlusive agents, emollients, thickeners, solubilizing agents, penetration enhancers, anti-irritants, colorants, propellants (as described above) and surfactants.

Thus, for example, the compositions of the present invention can comprise, in combination with ammonium lactate and urea, one or more additional humectants or moisturizing agents. Representative examples of humectants that are usable in this context of the present invention include, without limitation, guanidine, glycolic acid and glycolate salts (e.g. ammonium slat and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

The compositions of the present invention can further comprise a pH adjusting agent. As is discussed hereinabove, although the ammonium lactate or any corresponding ammonium salt may serve as a pH adjusting agent, it is preferable for the compositions of the invention to have a pH value of between about 4 and about 7, preferably between about 5 and about 6, most preferably about 5.5 or substantially 5.5 and hence the presence of a pH adjusting agent is preferred. Suitable pH adjusting agents include, for example, one or more of adipic acids, glycines, citric acids, calcium hydroxides, magnesium aluminometasilicates, buffers or any combinations thereof.

Representative examples of deodorant agents that are usable in the context of the present invention include, without limitation, quaternary ammonium compounds such as cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmlthyl sarcosine, lauroyl sarcosine, N-myristoyl glycine, potassium N-lauryl sarcosine, stearyl, trimethyl ammonium chloride, sodium aluminum chlorohydroxy lactate, tricetylmethyl ammonium chloride, 2,4,4′-trichloro-2′-hydroxy diphenyl ether, diaminoalkyl amides such as L-lysine hexadecyl amide, heavy metal salts of citrate, salicylate, and piroctose, especially zinc salts, and acids thereof, heavy metal salts of pyrithione, especially zinc pyrithione and zinc phenolsulfate. Other deodorant agents include, without limitation, odor absorbing materials such as carbonate and bicarbonate salts, e.g. as the alkali metal carbonates and bicarbonates, ammonium and tetraalkylammonium carbonates and bicarbonates, especially the sodium and potassium salts, or any combination of the above.

Antiperspirant agents can be incorporated in the compositions of the present invention either in a solubilized or a particulate form and include, for example, aluminum or zirconium astringent salts or complexes.

Representative examples of sun screening agents usable in context of the present invention include, without limitation, p-aminobenzoic acid, salts and derivatives thereof (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one) and 4-isopropyl-di-benzoylmethane, and any combination thereof.

Representative examples of sunless tanning agents usable in context of the present invention include, without limitation, dihydroxyacetone, glyceraldehyde, indoles and their derivatives. The sunless tanning agents can be used in combination with the sunscreen agents.

Suitable hair conditioning agents that can be used in the context of the present invention include, for example, one or more collagens, cationic surfactants, modified silicones, proteins, keratins, dimethicone polyols, quaternary ammonium compounds, halogenated quaternary ammonium compounds, alkoxylated carboxylic acids, alkoxylated alcohols, alkoxylated amides, sorbitan derivatives, esters, polymeric ethers, glyceryl esters, or any combinations thereof.

The chelating agents are optionally added to the compositions of the present invention so as to enhance the preservative or preservative system. Preferred chelating agents are mild agents, such as, for example, ethylenediaminetetraacetic acid (EDTA), EDTA derivatives, or any combination thereof.

Suitable preservatives that can be used in the context of the present composition include, without limitation, one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or any combinations thereof.

Suitable emulsifiers that can be used in the context of the present invention include, for example, one or more sorbitans, alkoxylated fatty alcohols, alkylpolyglycosides, soaps, alkyl sulfates, monoalkyl and dialkyl phosphates, alkyl sulphonates, acyl isothionates, or any combinations thereof.

Suitable occlusive agents that can be used in the context of the present invention include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Suitable emollients, other than ammonium lactate, that can be used in the context of the present invention include, for example, dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Suitable thickeners that can be used in the context of the present invention include, for example, non-ionic water-soluble polymers such as hydroxyethylcellulose (commercially available under the Trademark Natrosol.RTM. 250 or 350), cationic water-soluble polymers such as Polyquat 37 (commercially available under the Trademark Synthalen.RTM. CN), fatty alcohols, fatty acids and their alkali salts and mixtures thereof.

Representative examples of solubilizing agents that are usable in this context of the present invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEENS and spans, e.g., TWEEN 80. Other solubilizers that are usable for the compositions of the present invention are, for example, polyoxyethylene sorbitan fatty acid ester, polyoxyethylene n-alkyl ethers, n-alkyl amine n-oxides, poloxamers, organic solvents, phospholipids and cyclodextrines.

Suitable penetration enhancers usable in context of the present invention include, but are not limited to, dimethylsulfoxide (DMSO), dimethyl formamide (DMF), allantoin, urazole, N,N-dimethylacetamide (DMA), decylmethylsulfoxide (C₁₀ MSO), polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark Azone^{RTM} from Whitby Research Incorporated, Richmond, Va.), alcohols, and the like. The permeation enhancer may also be a vegetable oil. Such oils include, for example, safflower oil, cottonseed oil and corn oil.

Suitable anti-irritants that can be used in the context of the present invention include, for example, steroidal and non steroidal anti-inflammatory agents or other materials such as aloe vera, chamomile, alpha-bisabolol, cola nitida extract, green tea extract, tea tree oil, licoric extract, allantoin, caffeine or other xanthines, glycyrrhizic acid and its derivatives.

Although a wide variety of ingredients can be included in the compositions of the present invention, in addition to the active ingredients, the compositions are preferably devoid of an enduring perfume composition. The incorporation of such a perfume composition in pharmaceutical compositions is considered in the art disadvantageous for skin and scalp medical treatment, as it oftentimes cause undesirable irritation of a sensitive skin.

As used herein, the phrase “an enduring perfume composition” describes a composition that comprises one or more perfumes that provide a long lasting aesthetic benefit with a minimum amount of material. Enduring perfume compositions are substantially deposited and remain on the body throughout any rinse and/or drying steps. Representative examples of such compositions are described, for example, in U.S. Pat. No. 6,086,903.

However, it should be noted that fragrances other than enduring perfume compositions, perfumes or perfume compositions, which are fast removable from the surface they are deposited on, can be included in the compositions of the present invention.

Further optionally, the compositions of the present invention can comprise, in addition to the urea and/or the derivative thereof and ammonium lactate or any other alpha-hydroxy acid or a salt thereof, one or more other active ingredients (also referred to herein as “additional active ingredient(s)”), which are aimed at providing the composition with an additional therapeutic, cosmeceutic or cosmetic effect.

As is described hereinabove, the phrase “active ingredient” refers to an ingredient which exerts a pharmacological, dermatological, cosmetic, cosmeceutical or any other beneficial activity.

Compositions that include additional active ingredient(s) may therefore be efficiently used in the treatment of skin and/or scalp medical, cosmetic and cosmeceutical conditions other than dry skin and scalp, such as, for example, infections, fungi, allergies, aging and more.

Preferred additional active ingredients according to the present invention include, without limitation, one or more, or any combination of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an anti-dandruff agent.

Suitable anti-acne agents for use in this context of the present invention include, without limitation, keratolytics such as salicylic acid, sulfur, glycolic, pyruvic acid, resorcinol, and N-acetylcysteine and retinoids such as retinoic acid and its derivatives (e.g., cis and trans, esters).

Suitable antibiotics for use in this context of the present invention include, without limitation, benzoyl peroxide, octopirox, erythromycin, zinc, tetracyclin, triclosan, azelaic acid and its derivatives, phenoxy ethanol and phenoxy proponol, ethylacetate, clindamycin and meclocycline; sebostats such as flavinoids; alpha and beta hydroxy acids; and bile salts such as scymnol sulfate and its derivatives, deoxycholate and cholate.

Representative examples of non-steroidal anti-inflammatory agents that are usable in this context of the present invention include, without limitation, oxicams, such as piroxicam, isoxicam, tenoxicam, sudoxicam, and CP-14,304; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents. For example, etofenamate, a flufenamic acid derivative, is particularly useful for topical application.

Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Suitable antipruritic agents include, without limitation, pharmaceutically acceptable salts of methdilazine and trimeprazine.

Non-limiting examples of anesthetic drugs that are suitable for use in context of the present invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

Suitable antimicrobial agents, including antibacterial, antifungal, antiprotozoal and antiviral agents, for use in context of the present invention include, without limitation, beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, farnesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate and clotrimazole and mixtures thereof.

Non-limiting examples of anti-oxidants that are usable in the context of the present invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox^{R}), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Non-limiting examples of chemotherapeutic agents usable in context of the present invention include daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A and XR9576.

Non-limiting examples of antidepressants usable in context of the present invention include norepinephrine-reuptake inhibitors (“NRIs”), selective-serotonin-reuptake inhibitors (SSRIs), monoamine-oxidase inhibitors (MAOIs), serotonin-and-noradrenaline-reuptake inhibitors (“SNFIs), corticotropin-releasing factor (CRF) antagonists, α-adrenoreceptor antagonists, NK1-receptor antagonists, 5-HT_{1A}-receptor agonist, antagonists, and partial agonists and atypical antidepressants, as well as norepinephrine-reuptake inhibitors such as, but are not limited to amitriptyline, desmethylamitriptyline, clomipramine, doxepin, imipramine, imipramine-oxide, trimipramine; adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norclolipramine, noxiptilin, opipramol, perlapine, pizotyline, propizepine, quinupramine, reboxetine, tianeptine, and serotonin-reuptake inhibitors such as, but are not limited to, binedaline, m-chloropiperzine, citalopram, duloxetine, etoperidone, femoxetine, fluoxetine, fluvoxamine, indalpine, indeloxazine, milnacipran, nefazodone, oxaflazone, paroxetine, prolintane, ritanserin, sertraline, tandospirone, venlafaxine and zimeldine.

Exemplary anti-dandruff ingredients usable in context of the present invention include, without limitation, zinc pyrithione, shale oil and derivatives thereof such as sulfonated shale oil, selenium sulfide, sulfur; salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazolenitrite and any possible stereo isomers and derivatives thereof such as anthralin, piroctone olamine (Octopirox), selenium sulfide, and ciclopirox olamine, and mixtures thereof.

Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B₃ (niacinamide and its derivatives), alpha hydroxy acids (such as glycolic acid, lactic acid, tartaric acid, malic acid, citric acid, etc.) and beta hydroxy acids (such as salicylic acid and the like).

Non-limiting examples of dermatological active ingredients usable in context of the present invention include jojoba oil and aromatic oils such as methyl salicylate, wintergreen, peppermint oil, bay oil, eucalyptus oil and citrus oils, as well as ammonium phenolsulfonate, bismuth subgallate, zinc phenolsulfonate and zinc salicylate. Non-limiting examples of antifungal agents include miconazole, clotrimazole, butoconazole, fenticonasole, tioconazole, terconazole, sulconazole, fluconazole, haloprogin, ketonazole, ketoconazole, oxinazole, econazole, itraconazole, terbinafine, nystatin and griseofulvin.

Non-limiting examples of antihistamines usable in context of the present invention include chlorpheniramine, brompheniramine, dexchlorpheniramine, tripolidine, clemastine, diphenhydramine, promethazine, piperazines, piperidines, astemizole, loratadine and terfenadine.

Suitable hormones for use in the context of the present invention include, for example, androgenic compounds and progestin compounds.

Representative examples of androgenic compounds include, without limitation, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone and pharmaceutically acceptable esters and salts thereof, and combinations of any of the foregoing.

Representative examples of progestin compounds include, without limitation, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-3β-ol-20-one, 4-pregnen-20β-ol-3-one-20-sulfate, acetoxypregnenolone, anagestone acetate, cyproterone, dihydrogesterone, fluorogestone acetate, gestadene, hydroxyprogesterone acetate, hydroxymethylprogesterone, hydroxymethyl progesterone acetate, 3-ketodesogestrel, megestrol, melengestrol acetate, norethisterone and mixtures thereof.

The compositions of the present invention may be packed or presented in any convenient way. For example, they may be packed in a tube, a bottle, or a pressurized container, using techniques well known to those skilled in the art and as set forth in reference works such as Remington's Pharmaceutical Science 15^{th} Ed. It is preferred that the packaging is done in such a way so as to minimize contact of the unused compositions with the environment, in order to minimize contamination of the compositions before and after the container is opened.

As the compositions of the present invention preferably include urea and ammonium lactate, and/or any related substances as is detailed hereinabove, as active ingredients, these compositions are useful in preventing or treating medical or cosmetic conditions associated with dry skin and/or scalp such as, for example, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, and xeroderma pigmentosum.

Hence, in a preferred embodiment of the present invention, each of the compositions described hereinabove, is packaged in a packaging material and is identified in print, in or on the package, for use in the treatment or prevention of dry skin and/or scalp and/or any one or more of the conditions listed or described herein.

The efficacy of the compositions of the present invention in treating conditions associated with dry skin and scalp is well demonstrated in the Examples section that follows.

Hence, according to another aspect of the present invention, there is provided a method of treating a medical and/or cosmetic condition associated with dry skin and/or scalp. The method is effected by topically applying onto an affected biological surface, e.g., a dry skin and/or scalp, a pharmaceutically, cosmetically or cosmeceutically effective amount of any of the compositions of the present invention as described herein.

As is described hereinabove, the compositions of the present invention can include, in addition to urea, ammonium lactate and/or the related substances detailed hereinabove, additional active ingredients, which exert therapeutic, cosmetic and/or cosmeceutical activities other than hydration. The additional active ingredients therefore render the compositions of the present invention useful in treating or preventing any medical, cosmetic and/or cosmeceutical condition of the skin and/or scalp.

Thus, the method, according to this aspect of the present invention, is further of treating any dermatological (e.g., of the skin and/or scalp) medical, cosmetic or cosmeceutical condition, other than dry skin and/or scalp, as is described hereinabove.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The phrase “topically applying” describes application onto one or more biological surface(s), e.g., skin or scalp, by direct laying or spreading a composition on the surface. Non-limiting examples of biological surfaces onto which the compositions of the present invention can be topically applied include one or more of the lateral aspect of forearms, the lateral aspect of legs, elbows, palms, feet, backhands, back, scalp and any other dry skin surface.

According to this aspect of the present invention, the compositions of the present invention are preferably topically applied between one and four times a day, more preferably twice a day (e.g., once in the morning and once in the evening). The topical application of the compositions of the present invention is preferably carried out for a time period that ranges between 1 and 30 days, more preferably for a time period of about fourteen days.

The phrase “pharmaceutically, cosmetically or cosmeceutically effective amount” describes an amount of a composition that is sufficient to significantly induce a positive modification in the condition being treated, but low enough to avoid significant side effects, within the scope of sound judgment of the skilled artisan. The effective amount of the composition may vary with the particular skin being treated, the age and physical condition of the biological subject being treated, the severity of the condition, the duration of the treatment, the nature of concurrent therapy, the specific compound, composition or other material employed, the particular pharmaceutically, cosmetically or cosmeceutically acceptable topical carrier utilized, and like factors within the knowledge and expertise of the skilled artisan.

According to another aspect of the present invention there is provided a process of preparing the novel compositions described hereinabove. The process generally comprises admixing the active ingredients described hereinabove and the pharmaceutically, cosmetically or cosmeceutically acceptable carrier. In cases were other agents or active agents, as is detailed hereinabove, are present in the compositions, the process includes admixing these agents together with the active ingredients and the carrier. The mixing technique utilized in the process of the present invention depends on the nature of the carrier, the desired form of the composition and the agents included in the composition. A variety of exemplary formulation techniques that are usable in the process of the present invention is described, for example, in Harry's Cosmeticology, Seventh Edition, Edited by JB Wilkinson and R J Moore, Longmann Scientific & Technical, 1982, Chapter 13 “The Manufacture of Cosmetics” pages 757-799. Preferably, a formulation technique that is usable within this aspect of the present invention typically involves mixing each of the active ingredients (e.g., urea and ammonium lactate) and the selected carrier concomitantly, namely, as a one-pot procedure.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Skin and Scalp Compositions

Representative examples of skin and scalp topical compositions, according to the present invention, were prepared in various forms using conventional methods (see, for example, Harry's Cosmeticology, Seventh Edition, Edited by JB Wilkinson and R J Moore, Longmann Scientific & Technical, 1982, Chapter 13 “The Manufacture of Cosmetics” pages 757-799), whereby each of these representative compositions comprise 20 weight percentages (% wt.) urea and 12 weight percentages of ammonium lactate, obtained using 17 weight percentages of a 70% ammonium lactate source solution. The components of each of these compositions are listed hereinbelow:

Composition 1—A Skin Foam:

| | |
|---|---|
| GLYCERINE | 2.0% wt. |
| ALLANTOIN | 0.2% wt. |
| UREA USP | 20% wt. |
| CETYL ALCOHOL | 0.5% wt. |
| VASELINE ™ | 8.0% wt. |
| Isopropyl myristate | 4.0% wt. |
| MYRITOL 318 ™ | 3.0% wt. |
| TWEEN 60 ™ | 1.0% wt. |
| SILICON D.C.350 ™ | 0.5% wt. |
| VIT.E ACETATE | 0.1% wt. |
| MONTANOV 68 ™ | 1.5% wt. |
| PHENONIP ™ | 0.7% wt. |
| AMYLUM RICE STARCH ™ | 2.0% wt. |
| PURASAL NH 70 ™* | 17% wt. |
| DMDM HYDANTOIN ™ | 0.35% wt. |
| WATER | qs 100% |

*A 70% solution of ammonium lactate

Composition 2—A Scalp Foam Shampoo:

| MERQUAT 550 ™ (Polyquaternium-7) | 1.0% wt. |
|---|---|
| ALLANTOIN | 0.2% wt. |
| PURASAL NH 70 ™* | 17.0% wt. |
| UREA USP | 20.0% wt. |
| Ammonium Laureth Sulfate - 25% | 15.0% wt. |
| Cocamidopropyl Betaine - 35% | 4.0% wt. |
| DMDM Hydantoin ™ | 0.35% wt. |
| Perfume IFF GOGO 3787 ™ | 0.1% wt. |
| WATER | qs 100% |
| PH | 5.5 |

*A 70% solution of ammonium lactate

Composition 3—A Scalp Shampoo:

| MERQUAT 550 ™ (Polyquaternium-7) | 1.0% wt. |
|---|---|
| ALLANTOIN | 0.2% wt. |
| PURASAL NH 70 ™* | 17.0% wt. |
| UREA USP | 20.0% wt. |
| Ammonium Laureth Sulfate - 25% | 25.0% wt. |
| Cocamidopropyl Betaine - 35% | 5.0% wt. |
| DMDM Hydantoin ™ | 0.35% wt. |
| Perfume IFF GOGO 3787 ™ | 0.1% wt. |
| WATER | qs 100% |
| PH | 5.5 |

*A 70% solution of ammonium lactate

Composition 4—A Cream:

| GLYCERINE | 2.0% wt. |
|---|---|
| ALLANTOIN | 0.2% wt. |
| UREA USP | 20% wt. |
| CETYL ALCOHOL | 3.0% wt. |
| VASELINE ™ | 4.0% wt. |
| RHODICARE D ™ | 0.1% wt. |
| MYRITOL 318 ™ | 3.0% wt. |
| CRODAMOL OP ™ | 4.0% wt. |
| SILICON D.C.350 ™ | 0.5% wt. |
| VIT.E ACETATE | 0.1% wt. |
| MONTANOV 68 ™ | 1.0% wt. |
| PHENONIP ™ | 0.7% wt. |
| AMYLUM RICE STARCH ™ | 2.0% wt. |
| PURASAL NH 70 ™* | 17% wt. |
| DMDM HYDANTOIN ™ | 0.35% wt. |
| POLYSINLAN ™ | 3.0% wt. |
| DRACORIN 100 SEP ™ | 2.0% wt. |
| PARF. LONI ™ | 0.15% wt. |
| Water | qs 100% |
| PH | 5.5 |

*A 70% solution of ammonium lactate

Composition 5—A Clear Gel:

| BRONOPOL ™ | 0.02% wt. |
|---|---|
| NATROSOL 250 HHX ™ | 1.0% wt. |
| ALLANTOIN | 0.2% wt. |
| GLYCERINE | 3.0% wt. |
| UREA | 20% wt. |
| PURASAL NH-70 ™* | 17% wt. |
| Water | qs 100% |
| PH | 5.5 |

*A 70% solution of ammonium lactate

Composition 6—A Scalp Spray-Gel:

| | |
|---|---|
| CELQUAT H-100 ™ | 1.0% wt. |
| TAGAT R-40 ™ | 1.0% wt. |
| ALLANTOIN | 0.2% wt. |
| GLYCERINE | 2.0% wt. |
| UREA | 20% wt. |
| PURASAL NH-70 ™* | 17% wt. |
| PARF. LONI ™ | 0.1% wt. |
| DMDM Hydantoin ™ | 0.35% wt. |
| TWEEN 20 ™ | 0.2% wt. |
| Water | qs 100% |

*A 70% solution of ammonium lactate

Representative examples of additional skin and scalp topical compositions, according to the present invention, which comprise 20 weight percentages (% wt.) urea and 14 weight percentages of ammonium lactate, obtained using 20 weight percentages of a 70% ammonium lactate source solution, are prepared using the methods described above and include the following components:

Composition 7—A Cream:

| | |
|---|---|
| GLYCERINE | 2.0% wt. |
| ALLANTOIN | 0.2% wt. |
| UREA USP | 20% wt. |
| CETYL ALCOHOL | 3.0% wt. |
| VASELINE ™ | 4.0% wt. |
| RHODICARE D ™ | 0.1% wt. |
| MYRITOL 318 ™ | 3.0% wt. |
| CRODAMOL OP ™ | 4.0% wt. |
| SILICON D.C.350 ™ | 0.5% wt. |
| VIT.E ACETATE | 0.1% wt. |
| MONTANOV 68 ™ | 1.0% wt. |
| PHENOXYETHANOL | 0.9% wt. |
| POTASSIUM SORBATE | 0.2% wt. |
| AMYLUM RICE STARCH ™ | 2.0% wt. |
| PURASAL NH 70 ™* | 20% wt. |
| Water | qs 100% |

*A 70% solution of ammonium lactate

Composition 8—A Mousse:

| GLYCERINE | 2.0% wt. |
|---|---|
| ALLANTOIN | 0.2% wt. |
| UREA USP | 20% wt. |
| CETYL ALCOHOL | 0.5% wt. |
| VASELINE ™ | 8.0% wt. |
| Isopropyl Myristate | 4.0% wt. |
| MYRITOL 318 ™ | 3.0% wt. |
| TWEEN 60 ™ | 1.0% wt. |
| SILICON D.C.350 ™ | 0.5% wt. |
| VIT.E ACETATE | 0.1% wt. |
| MONTANOV 68 ™ | 1.5% wt. |
| PHENONIP ™ | 0.7% wt. |
| AMYLUM RICE STARCH ™ | 2.0% wt. |
| PURASAL NH 70 ™* | 20% wt. |
| DMDM HYDANTOIN ™ | 0.35% wt. |
| Water | qs 100% |
| PH | 5.5 |

*A 70% solution of ammonium lactate

### Example 2

### Comparative Activity Tests

The activity of the compositions of the present invention in the treatment of dry skin was tested and compared with that of a commercially available dry skin product, containing ammonium lactate only. Thus, a skin foam composition that comprises 20% urea and 12% ammonium lactate (Composition 1 hereinabove) was tested for its activity in the treatment of dry skin according to the protocol described hereinbelow, whereby an Ammonium Lactate 12% cream, marketed by Clay Park Ltd., served as control.

Protocol: The study subjects were healthy volunteers, apparently free of diseases, aged 18 to 65, and having dry skin. The subjects had no history of skin or topical diseases and had no known sensitivity to any of the tested substances or to other components in the tested compositions.

Each volunteer was assigned a serial number by ISR.

Six volunteers that suffered from dry skin applied the tested preparations twice a day, on a daily basis for two weeks, once in the morning and once in the evening, on the lateral aspect of the forearms and legs, as follows: A small amount of the tested preparation was applied to clean hands. The preparation was rubbed/massaged into the forearm and legs with circular motion until it was absorbed into the skin. The composition of the present invention (Composition 1) was applied on the lateral part of the left leg and left forearm. The control composition was applied on the lateral part of the right leg and the right forearm.

Each volunteer of the trial was examined by a certified examiner prior to the start of the trial (baseline), at the end of the trial (two weeks later), and a week after the end of the trial (three weeks following baseline). The volunteer's condition was documented by photography and description of the appearance of the skin before and after applying the compositions. The results were used to calculate the SRRC index, which is the sum of the values of the following four indices: Scale, Roughness, Redness and Cracks. The results were graded according to the following scale:

0=absent

1=slight

2=moderate

3=severe

4=extreme,

and were thereafter summarized and converted to percentages of improvement.

In addition to visual evaluation, objective measurements were performed:

The skin hydration of the horny epidermal layer (stratum corneum) was evaluated by capacitance, using the Corneometer CM 820 PC capacitance meter (Courage & Khazaka). The capacitance of the horny epidermal layer increases with water content. The probe head (7×7 mm), consisting of a condenser, was applied to the skin surface at constant pressure. Recordings were performed in the laboratory room at ambient temperature of 20-23° C. and constant humidity. Doors and windows were kept closed. Participants were asked to refrain from walking and talking 15 minutes prior to the measurements. Each participant was examined by a certified examiner prior to the start of the trial, at the end of the trial (two weeks later), and a week after the end of the trial (three weeks following baseline).

The transepidermal water loss (TEWL) was measured with the TEWAMETER 210 (Courage & Khazaka), an electronic measuring device used to evaluate the water pressure gradient above the skin. The measurements were performed by a certified examiner prior to the start of the trial, at the end of the trial (two weeks later), and a week after the end of the trial (three weeks following baseline). The probe head of the device, consisting of two hydrosensors at different heights, was applied to the skin surface at constant pressure. Recordings were performed at ambient temperature of 20-23° C. and constant humidity. Doors and windows were kept closed. Participants were asked to refrain from walking and talking 15 minutes prior to the measurements. The range and average values of each measurement following application of the tested compositions were recorded and compared with the value measured before application.

Along with the above measurements, the participants completed a questionnaire querying how many years they had suffered from dry skin, which factors aggravated their condition, which factors improved their condition, prior use of preparations and their response to those preparations. The participants were also asked to indicate whether they had previous experience with compositions manufactured by the company under test.

In addition, the participants completed a questionnaire on their satisfaction with each of the tested compositions. The participants were asked to rank, on a scale from 0 to 10, the improvement in the dryness and itching parameters. The results were thereafter summarized and converted to percentages of improvement.

Additionally, the volunteers were asked to keep a record of side effects (if any), and to report them to ISR staff. This included dryness, burning, peeling, redness and the like. When necessary, the volunteer was examined by the dermatologist.

Results:

The results obtained from the volunteers assessments of the improvement in dryness and itching, expressed by percentages of improvement, are presented in Table 1 below.

TABLE 1

| | | Itching | | Dryness | |
|---|---|---|---|---|---|
| | | Composition 1 | Control | Composition 1 | Control |
| Forearm | A | 100 | 100 | 58 | 44 |
| | B | 100 | 100 | 65 | 56 |
| Leg | A | 100 | 100 | 64 | 56 |
| | B | 100 | 100 | 42 | 50 |

A = after two weeks of treatment
B = after two weeks of treatment and one week without treatment.

These results show that while the itching was 100% improved upon application of both the composition of the present invention, Composition 1, and the commercially available ammonium lactate preparation, denoted as the control, the improvement in the dryness parameter using the composition of the present invention was superior to the control, thus demonstrating the superior efficacy of the composition of the present invention in treating dry skin.

The results from the objective SRRC indices (evaluated by the examiner), expressed by percentages of improvement, are summarized in Table 2 below.

TABLE 2

| | | Cracks | | Roughness | | Redness | | Scale | |
|---|---|---|---|---|---|---|---|---|---|
| | | Composition 1 | control | Composition 1 | control | Composition 1 | control | Composition 1 | control |
| Forearm | A | No measurements were | | 100 | 80 | 80 | 83 | No measurements were | |
| | B | done on the forearms | | 80 | 100 | 100 | 83 | done on the forearms | |
| | | because there were no | | | | | | because there were no | |
| | | cracks in these areas | | | | | | scales in these areas | |
| Leg | A | 75 | 50 | 67 | 67 | 60 | 0 | 71 | 83 |
| | B | 50 | 50 | 67 | 67 | 60 | 40 | 43 | 33 |

A = after two weeks of treatment
B = after two weeks of treatment and one week without treatment.

These results show that in most of the tested parameters, the composition of the present invention, Composition 1, was superior to the commercially available control composition.

The results of obtained by the measuring devices are summarized in Table 3 below. These results show that the two tested compositions were nearly equal in the comeometry evaluation, while the composition of the present invention was found to be advantageous over the control, according to the TEWL measurements.

TABLE 3

| | | Corneometry | | TEWL | |
|---|---|---|---|---|---|
| | | (average improvement) | | (average rise) | |
| | | Composition 1 | control | Composition 1 | control |
| Forearm | A | 72 | 67 | 7 | 31 |
| | B | 32 | 35 | 25 | 56 |
| Leg | A | 44 | 50 | 9 | 13 |
| | B | 32 | 26 | 18 | 22 |

A = after two weeks of treatment
B = after two weeks of treatment and one week without treatment.

### Example 3

### Comparative Activity Tests

The activity of the compositions of the present invention in the treatment of dry skin was tested and compared with that of a commercially available dry skin product, containing urea only. Thus, a cream composition that comprises 20 weight percentages urea and 12 weight percentages ammonium lactate (Composition 4 hereinabove, also referred to herein as Moisturizing Complex Cream or MCC) was tested for its activity in the treatment of dry skin according to the protocol described hereinbelow, whereby an Urea 40% cream, marketed by Doak Dermatologics, a subsidiary of Bradley Pharmaceuticals Inc. (also referred to herein as Urea Cream or UC), served as control.

Protocol:

Fifteen (15) healthy volunteers, free of disease, aged 18 to 65, suffering from dry skin (xerosis) participated in this study.

The study consisted of a 14-days (two-weeks) treatment period in which a composition of the present invention (Composition 4, MCC) was applied to the outer side of the right forearm and right lower leg, and an Urea 40% Cream (control, UC) was applied to the outer side of the left forearm and left lower leg. The tested and the control compositions were applied twice a day and observations were made on day 0 (baseline), day 14, and, at the follow-up, on day 28.

Efficacy Evaluation was based on instrument measurements, investigator's clinical observations (objective criteria) and volunteers' (subjects') self-assessment (subjective criteria), as follows:

Instrument Measurements:

Water content of the stratum corneum was evaluated by the corneometer (CM 825, Courage & Khazaka). The probe of the corneometer was placed on the skin at constant pressure and measurements of skin hydration were taken. Skin hydration was recorded at baseline (day 0), day 14 and day 28. Room temperature and humidity were kept constant (20° C.-23° C. and 40%-50%, respectively). The tested subjects rested for 15 minutes before each measurement.

Transepidermal water loss (TEWL) was measured with an evaporimeter (Tewameter 300, Courage & Khazaka). TEWL was recorded at baseline, day 14 and day 28. Room temperature and humidity were kept constant (20° C.-23° C. and 40%-50%, respectively). The tested subjects rested for 15 minutes before each measurement.

Investigators' Assessment:

Investigators' evaluations were made on the following parameters: Scaling, Roughness, Redness, and Cracks (SRRC).

These parameters were evaluated on a scale of 0 to 4 where:

0=Absent

1=Slight

2=Moderate

3=Severe

4=Extreme

Subjective Measurements:

Subjects (participants) self-assessed two parameters—dryness and itchiness. These parameters were evaluated on a scale of 0 to 10.

The participants were also asked to complete a preference questionnaire at baseline and at the end of day 14, on which they were asked about the smell of the tested compositions, the absorption thereof and the effect thereof on skin texture.

Statistics:

Results were summarized in tables and graphs showing means and standard deviations. Special attention was given to the difference (expressed by improvement/deterioration) in the parameters as recorded by the subjective and objective measurements.

Results were analyzed using the nonparametric test-Wilcoxon signed-ranks test for hypotheses of an unknown distribution.

Results:

Objective Measurements:

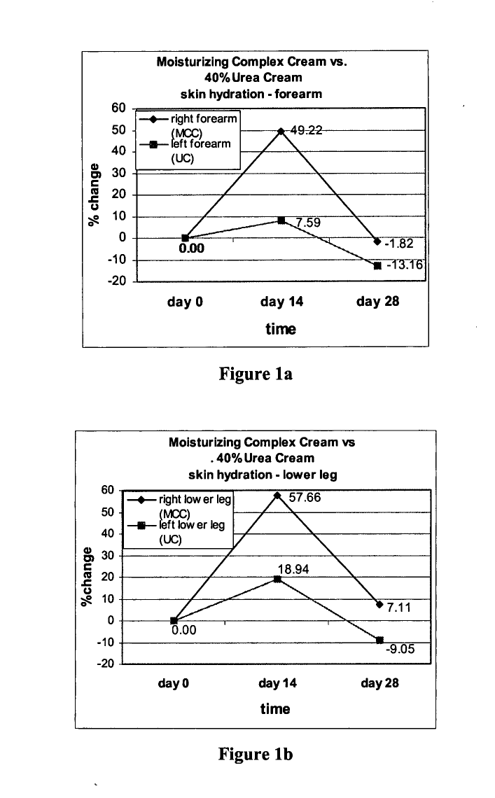

The results obtained by the measuring devices (corneometer and TEWL) are summarized in Table 4 below. The effect of the tested compositions of skin hydration is further presented in FIGS. 1a and 1b.

As is clearly demonstrated in FIG. 1a-b, the corneometer findings indicate that the composition of the present invention (Composition 4 hereinabove, denoted as MCC) was substantially more effective than the commercially available urea cream (denoted as UC) at skin hydration. After two weeks of treatment, MCC increased the capacitance value on the forearm by 50% and on the lower leg by 57%, while upon treatment with UC, the capacitance value was increased by 8% on the forearm and 19% on the lower leg. The effectiveness of MCC at skin hydration was statistically significant (p<0.05). On day 28 (follow-up visit), MCC still showed statistically significant improvement of this parameter, as compared with UC (p<0.05).

As is shown in Table 4, according to TEWL measurements on the forearm, the composition of the present invention (MCC) maintained a stable transepidermal water loss during the 2 weeks treatment period, as well as on day 28, while the commercial urea composition (UC) exhibited higher water loss.

TEWL measurements on the lower leg showed high water loss with both compositions during the treatment period and at follow-up on day 28. This can be explained by the extreme weather changes that occurred during the study, which involved substantially lower temperatures and accelerated water loss and skin dryness. In addition, TEWL is typically the appropriate tool for measuring dry skin in cases where there is clear and sufficient barrier damage, such as atopic dermatitis, and hence may not serve as an accurate measurement tool for this study.

TABLE 4

| | | Composition 4 (MCC) | | | Control (UC) | | |
|---|---|---|---|---|---|---|---|
| Measurement | | day 0 | day 14 | day 28 | day 0 | day 14 | day 28 |
| Skin Hydration | | | | | | | |
| Corneometer | forearm | 33.89 ± 5.76 | 47.89 ± 9.53 | 32.09 ± 5.80 | 35.38 ± 3.88 | 35.92 ± 5.53 | 30.16 ± 5.19 |
| (a.u.**) | lower leg | 29.04 ± 7.15 | 40.19 ± 14.43 | 30.46 ± 6.90 | 29.89 ± 9.98 | 33.61 ± 11.35 | 26.10 ± 6.89 |
| Average | forearm | | 49.22 ± 49.72 | −1.82 ± 31.56 | | 7.59 ± 53.55 | −13.16 ± 15.38 |
| Improvement* (%) | lower leg | | 57.66 ± 102.65 | 7.11 ± 26.91 | | 18.94 ± 37.33 | −9.05 ± 21.02 |
| TEWL | | | | | | | |
| Evaporimeter | forearm | 7.54 ± 1.36 | 7.77 ± 1.93 | 7.49 ± 4.13 | 7.65 ± 2.24 | 10.07 ± 6.93 | 9.73 ± 5.40 |
| [g/(hm\hat{ }2)] | lower leg | 6.74 ± 1.16 | 7.96 ± 3.03 | 9.65 ± 2.16 | 7.02 ± 1.11 | 8.03 ± 1.47 | 9.71 ± 3.79 |
| Average | forearm | | 5.81 ± 22.19 | 0.17 ± 32.88 | | 26.19 ± 70.88 | 35.95 ± 63.27 |
| Improvement* (%) | lower leg | | 25.43 ± 47.46 | 48.81 ± 55.16 | | 23.58 ± 42.48 | 50.34 ± 69.03 |

*Due to the dependency between the measurements, results are shown as ratio change and not as average change.
**a.u. = auxiliary units

Investigators Assessment:

The results of the objective SRRC indices, evaluated by the investigators, are summarized in Table 5 below.

TABLE 5

| | | | Composition 4 (MCC) | | Control (UC) | |
|---|---|---|---|---|---|---|
| | | | Day 14 | Day 28 | Day 14 | Day 28 |
| | | Treated | No. (%) of | No. (%) of | No. (%) of | No. (%) of |
| Parameter | Change | Area | respondents | respondents | respondents | respondents |
| Scalines | Improved | forearm | 6 (40) | 5 (34) | 4 (27) | 2 (13) |
| | | lower leg | 12 (80) | 5 (34) | 11 (74) | 3 (20) |
| | Same | forearm | 6 (40) | 9 (60) | 11 (73) | 9 (60) |
| | | lower leg | 3 (20) | 8 (53) | 4 (27) | 9 (60) |
| | Deteriorated | forearm | 3 (20) | 1 (7) | 0 (0) | 4 (27) |
| | | lower leg | 0 (0) | 2 (13) | 0 (0) | 3 (20) |
| Roughness | Improved | Forearm | 6 (40) | 7 (47) | 4 (27) | 6 (40) |
| | | lower leg | 7 (47) | 7 (47) | 8 (53) | 6 (40) |
| | Same | forearm | 8 (53) | 6 (40) | 9 (60) | 5 (33) |
| | | lower leg | 7 (47) | 8 (53) | 3 (20) | 5 (33) |
| | Deteriorated | forearm | 1 (7) | 2 (13) | 2 (13) | 4 (27) |
| | | lower leg | 1 (7) | 0 (0) | 4 (27) | 4 (27) |
| Redness | Improved | forearm | 0 (0) | 2 (13) | 6 (40) | 2 (13) |
| | | lower leg | 1 (7) | 1 (7) | 5 (33) | 2 (13) |
| | Same | forearm | 11 (73) | 7 (47) | 6 (40) | 8 (53) |
| | | lower leg | 12 (80) | 13 (87) | 8 (53) | 10 (67) |
| | Deteriorated | forearm | 4 (27) | 6 (40) | 3 (20) | 5 (33) |
| | | lower leg | 2 (13) | 1 (7) | 2 (13) | 3 (20) |
| Cracks | Improved | forearm | 2 (13) | 2 (13) | 2 (13) | 2 (13) |
| | | lower leg | 10 (67) | 5 (34) | 10 (67) | 4 (27) |
| | Same | forearm | 12 (80) | 12 (80) | 13 (87) | 12 (80) |
| | | lower leg | 4 (27) | 8 (53) | 5 (33) | 7 (47) |
| | Deteriorated | forearm | 1 (7) | 1 (7) | 0 (0) | 1 (7) |
| | | lower leg | 1 (7) | 2 (13) | 0 (0) | 4 (27) |

Scaliness and Cracks: As is shown in Table 5, treatment with both MCC (Composition 4) and UC (control) decreased the parameters of scaling and cracks, with no significant difference between the two therapies. It should be noted that as most of the volunteers did not suffer from skin cracks before the treatment, no substantial improvement was recorded in this respect.

Redness: As is further shown in Table 5, UC was found to be more effective at decreasing the parameter of skin redness, although the differences were not statistically significant. On day 28, both treatments preserved the degree of redness that was recorded at baseline (day 0). In about 35% of the cases, there was an increase in skin redness, which is presumably attributed to the weather changes described above.

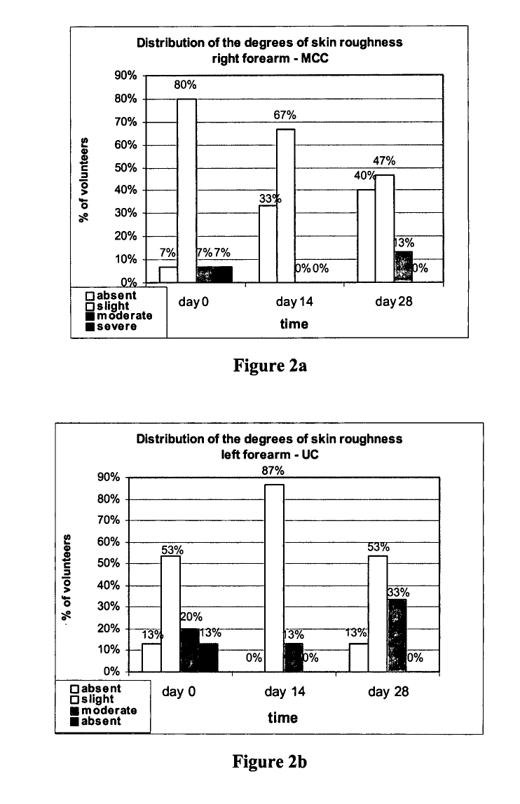

Roughness: Detailed evaluations of skin roughness in the forearms and lower legs are summarized in Tables 6-9 below and in FIGS. 2-5, as follows:

Table 6 below presents the investigators evaluations of the degree of skin roughness on the forearms of subjects treated with MCC (right forearm) and UC (left forearm), by time periods. The distribution of the degree of skin roughness is further presented in FIG. 2a (for MCC) and FIG. 2b (for UC).

TABLE 6

| Right Forearm (MCC) | | | | Left Forearm (UC) | | |
|---|---|---|---|---|---|---|
| Distribution | | | | Distribution | | |
| [No. of | | | | [No. of | | |
| respondents (%)] | | | Degree of | respondents (%)] | | |
| Day 0 | Day 14 | Day 28 | skin roughness | Day 0 | Day 14 | Day 28 |
| 1 (7) | 5 (33) | 6 (40) | 0 | 2 (13) | 0 (0) | 2 (13) |
| 12 (80) | 10 (67) | 7 (47) | 1 | 8 (53) | 13 (87) | 8 (53) |
| 1 (7) | 0 (0) | 2 (13) | 2 | 3 (20) | 2 (13) | 5 (33) |
| 1 (7) | 0 (0) | 0 (0) | 3 | 2 (13) | 0 (0) | 0 (0) |

As is shown in Table 6 and in FIGS. 2a-b, during the two-weeks treatment, both the composition of the present invention and the control composition decreased skin roughness on the forearms. However, wider improvement was recorded in subjects treated with MCC. For example, roughness was cleared in 40% of the forearms on which MCC was applied, compared with 13% of the forearms on which UC was applied.

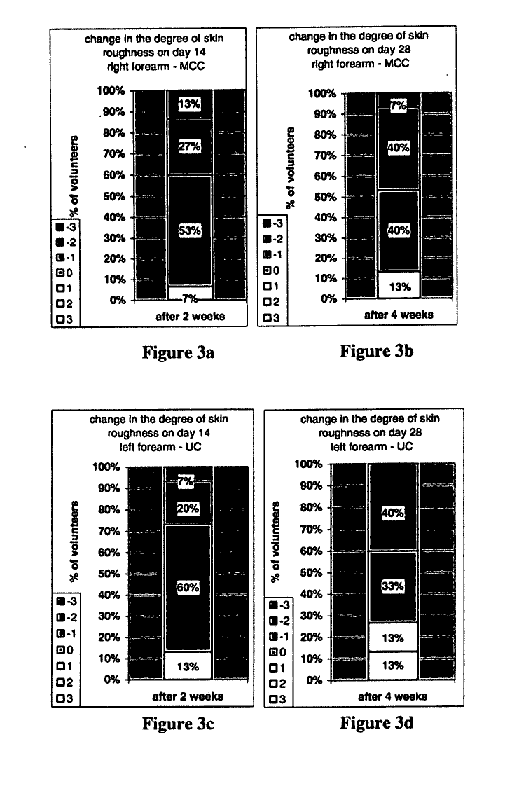

Table 7 below presents the investigators evaluations of the change of skin roughness on the forearms of subjects treated with MCC (right forearm) and UC (left forearm), after the two-weeks treatment and two additional weeks thereafter (4 weeks). The change in the degree of skin roughness after two and four weeks is further presented in FIGS. 3a and 3b (for MCC) and FIGS. 3c and 3d (for UC).

TABLE 7

| Right Forearm (MCC) | | | Left forearm (UC) | |
|---|---|---|---|---|
| Distribution | | | Distribution | |
| [No. of | | | [No. of | |
| respondents (%)] | | Change in | respondents (%)] | |
| After | After four | degree of skin | | After four |
| two weeks | weeks | roughness | After 2 weeks | weeks |
| 0 (0) | 0 (0) | 3 | 0 (0) | 0 (0) |
| 0 (0) | 0 (0) | 2 | 0 (0) | 2 (13) |
| 1 (7) | 2 (13) | 1 | 2 (13) | 2 (13) |
| 8 (53) | 6 (40) | 0 | 9 (60) | 5 (33) |
| 4 (27) | 6 (40) | −1 | 3 (20) | 6 (40) |
| 2 (13) | 1 (7) | −2 | 1 (7) | 0 (0) |
| 0 (0) | 0 (0) | −3 | 0 (0) | 0 (0) |

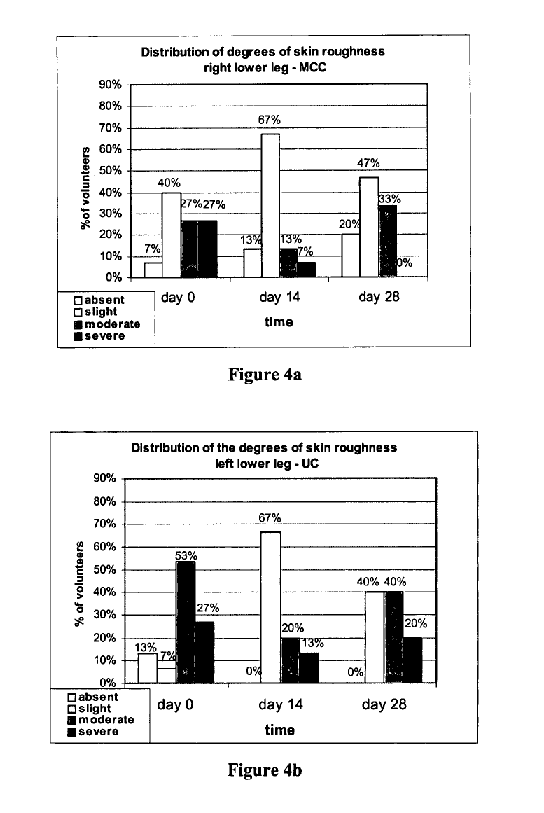

Table 8 below presents the investigators evaluations of the degree of skin roughness on the lower legs of subjects treated with MCC (right lower leg) and UC (left lower leg), by the time periods. The distribution of the degree of skin roughness is further presented in FIG. 4a (for MCC) and FIG. 4b (for UC).

As is shown in Table 8 and in FIGS. 4a-b, during the two-weeks treatment, both the composition of the present invention and the control composition decreased skin roughness on the lower legs. However, wider improvement was recorded in subjects treated with MCC. For example, on day 28, improvement was recorded for all the 27% of the volunteers who suffered from rough skin on the right lower leg (where MCC was applied) at baseline (day 0). Contrary to that, 20% of the participants who applied UC on the lower leg still suffered from skin roughness on day 28. Furthermore, 20% of the participants who used MCC enjoyed smooth skin (0 on the scale of roughness) on day 28, while none of those who used the UC were given a score of 0 on that scale.

TABLE 8

| Right Lower Leg (MCC) | | | | Left Lower Leg (UC) | | |
|---|---|---|---|---|---|---|
| Distribution | | | | Distribution | | |
| [No. of | | | Degree of | [No. of | | |
| respondents (%)] | | | skin | respondents (%)] | | |
| Day 0 | Day 14 | Day 28 | roughness | Day 0 | Day 14 | Day 28 |
| 1 (7) | 2 (13) | 3 (20) | 0 | 2 (13) | 0 (0) | 0 (0) |
| 6 (40) | 10 (67) | 7 (47) | 1 | 1 (7) | 10 (67) | 6 (40) |
| 4 (27) | 2 (13) | 5 (33) | 2 | 8 (53) | 3 (20) | 6 (40) |
| 4 (27) | 1 (7) | 0 (0) | 3 | 4 (27) | 2 (13) | 3 (20) |

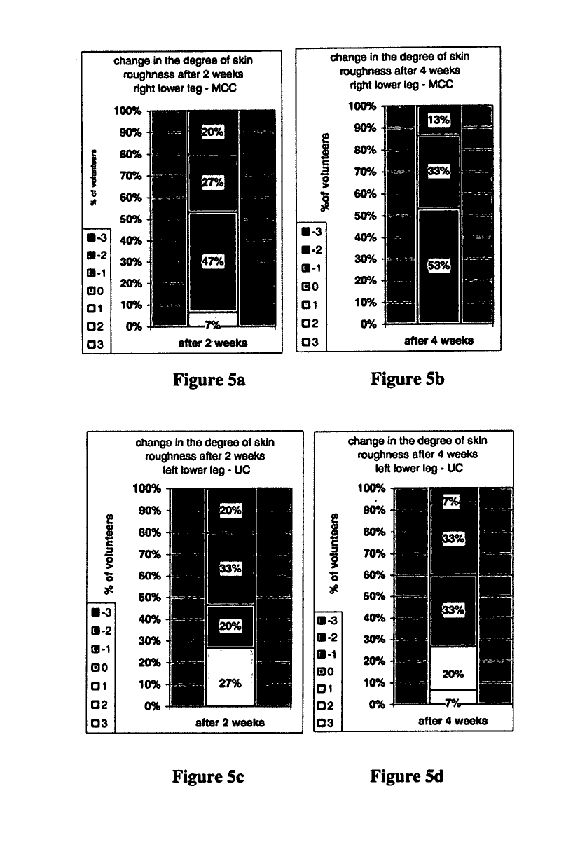

Table 9 below presents the investigators evaluations of the change of skin roughness on the lower legs of subjects treated with MCC (right lower leg) and UC (left lower leg), after the two-weeks treatment and two additional weeks thereafter (4 weeks). The change in the degree of skin roughness after two and four weeks is further presented in FIGS. 5a and 5b (for MCC) and FIGS. 5c and 5d (for UC).

TABLE 9

| Right Lower Leg (MCC) | | | Left Lower Leg (UC) | |
|---|---|---|---|---|
| Distribution | | | Distribution | |
| [No. of | | | [No. of | |
| respondents (%)] | | Change in | respondents (%)] | |
| After two | After four | degree of skin | | After four |
| weeks | weeks | roughness | After 2 weeks | weeks |
| 0 (0) | 0 (0) | 3 | 0 (0) | 0 (0) |
| 0 (0) | 0 (0) | 2 | 0 (0) | 1 (7) |
| 1 (7) | 0 (0) | 1 | 4 (27) | 3 (20) |
| 7 (47) | 8 (53) | 0 | 3 (20) | 5 (33) |
| 4 (27) | 5 (33) | −1 | 5 (33) | 5 (33) |
| 3 (20) | 2 (13) | −2 | 3 (20) | 1 (7) |
| 0 (0) | 0 (0) | −3 | 0 (0) | 0 (0) |

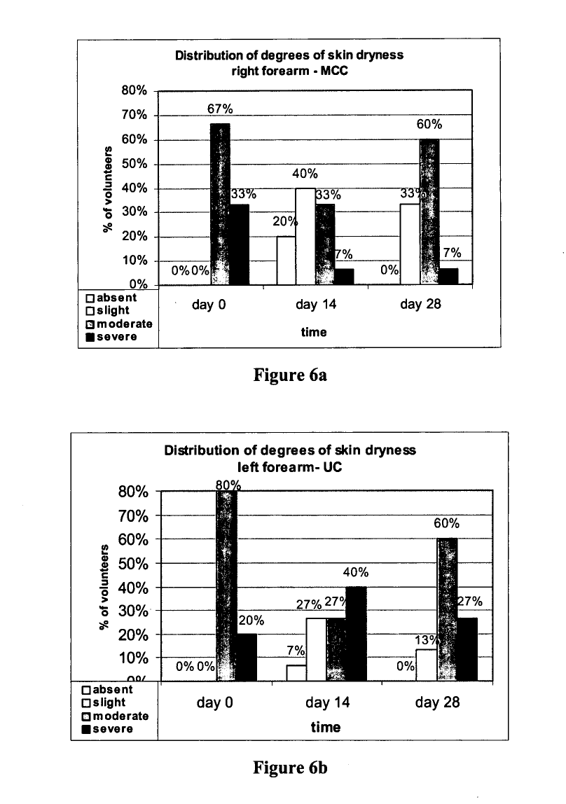

Skin Dryness: Detailed evaluations of skin dryness in the forearms and lower legs are summarized in Tables 10-13 below and in FIGS. 6-9, as follows:

Table 10 below presents the investigators evaluations of the degree of skin dryness on the forearms of subjects treated with MCC (right forearm) and UC (left forearm), by the time period. The distribution of the degree of skin dryness is further presented in FIG. 6a (for MCC) and FIG. 6b (for UC).

TABLE 10

| Right Forearm (MCC) | | | | Left Forearm (UC) | | |
|---|---|---|---|---|---|---|
| Distribution | | | | Distribution | | |
| [No. of | | | | [No. of | | |
| respondents (%)] | | | Degree of | respondents (%)] | | |
| Day 0 | Day 14 | Day 28 | skin dryness | Day 0 | Day 14 | Day 28 |
| 0 (0) | 3 (20) | 0 (0) | 0 | 0 (0) | 1 (7) | 0 (0) |
| 0 (0) | 6 (40) | 5 (33) | 1 | 0 (0) | 4 (27) | 2 (13) |
| 10 (67) | 5 (33) | 9 (60) | 2 | 12 (80) | 4 (27) | 9 (60) |
| 5 (33) | 1 (7) | 1 (7) | 3 | 3 (20) | 6 (40) | 4 (27) |

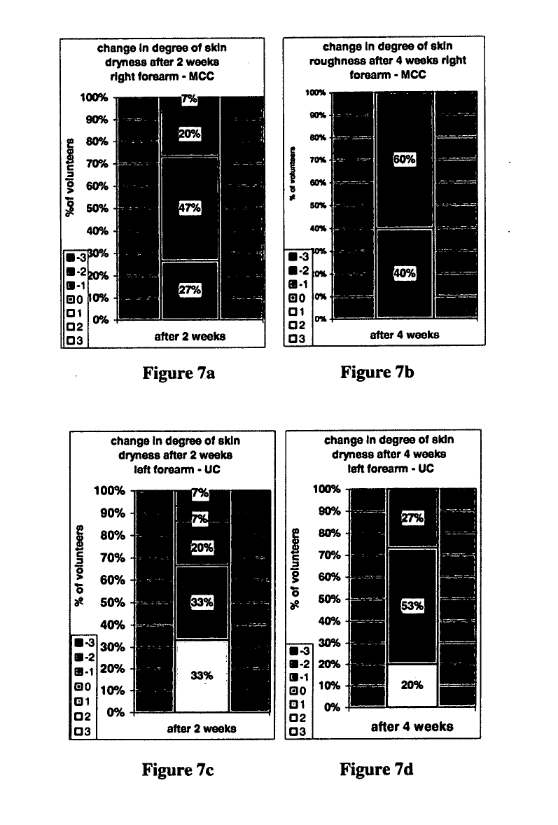

Table 11 below presents the investigators evaluations of the change in skin dryness on the forearms of subjects treated with MCC (right forearm) and UC (left forearm), after the two-weeks treatment and two additional weeks thereafter (4 weeks). The change in the degree of skin dryness after two and four weeks is further presented in FIGS. 7a and 7b (for MCC) and FIGS. 7c and 7d (for UC).

TABLE 11

| Right Forearm (MCC) | | | Left forearm (UC) | |
|---|---|---|---|---|
| Distribution | | | Distribution | |
| [No. of | | | [No. of | |
| respondents (%)] | | Change in | respondents (%)] | |
| After two | After four | degree of skin | | After four |
| weeks | weeks | dryness | After 2 weeks | weeks |
| 0 (0) | 0 (0) | 3 | 0 (0) | 0 (0) |
| 0 (0) | 0 (0) | 2 | 0 (0) | 0 (0) |
| 0 (0) | 0 (0) | 1 | 5 (33) | 3 (20) |
| 4 (27) | 6 (40) | 0 | 5 (33) | 8 (53) |
| 7 (47) | 9 (60) | −1 | 3 (20) | 4 (27) |
| 3 (20) | 0 (0) | −2 | 1 (7) | 0 (0) |
| 1 (7) | 0 (0) | −3 | 1 (7) | 0 (0) |

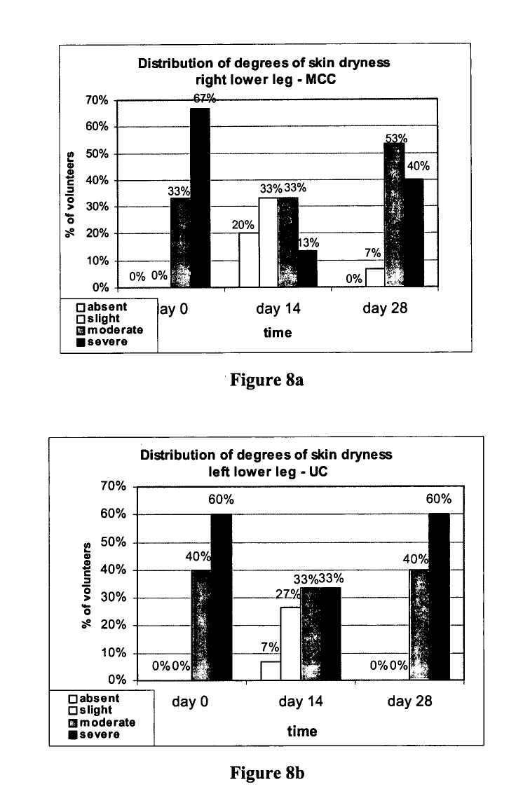

Table 12 below presents the investigators evaluations of the degree of skin on the lower legs of subjects treated with MCC (right lower leg) and UC (left leg), by the time periods. The distribution of the degree of skin dryness is presented in FIG. 8a (for MCC) and FIG. 8b (for UC).

TABLE 12

| Right Lower Leg (MCC) | | | | Left Lower Leg (UC) | | |
|---|---|---|---|---|---|---|
| Distribution | | | | Distribution | | |
| [No. of respondents (%)] | | | Degree of | [No. of respondents (%)] | | |
| Day 0 | Day 14 | Day 28 | skin dryness | Day 0 | Day 14 | Day 28 |
| 0 (7) | 3 (20) | 0 (0) | 0 | 0 (0) | 1 (7) | 0 (0) |
| 0 (0) | 5 (33) | 1 (7) | 1 | 0 (0) | 4 (27) | 0 (0) |
| 5 (33) | 5 (33) | 8 (53) | 2 | 6 (40) | 5 (33) | 6 (40) |
| 10 (67) | 2 (13) | 6 (40) | 3 | 9 (60) | 5 (33) | 9 (60) |

TABLE 13

| Right Lower Leg (MCC) | | | Left Lower Leg (UC) | |
|---|---|---|---|---|
| Distribution | | | Distribution | |
| [No. of respondents (%)] | | Change in | [No. of respondents (%)] | |
| After two | After four | degree of skin | | After four |
| weeks | weeks | dryness | After 2 weeks | weeks |
| 0 (0) | 0 (0) | 3 | 0 (0) | 0 (0) |
| 0 (0) | 0 (0) | 2 | 0 (0) | 0 (0) |
| 0 (0) | 0 (0) | 1 | 0 (0) | 2 (13) |
| 4 (27) | 10 (67) | 0 | 8 (53) | 11 (73) |
| 5 (33) | 5 (33) | −1 | 5 (33) | 2 (13) |
| 4 (27) | 0 (0) | −2 | 1 (7) | 0 (0) |
| 2 (13) | 0 (0) | −3 | 1 (7) | 0 (0) |

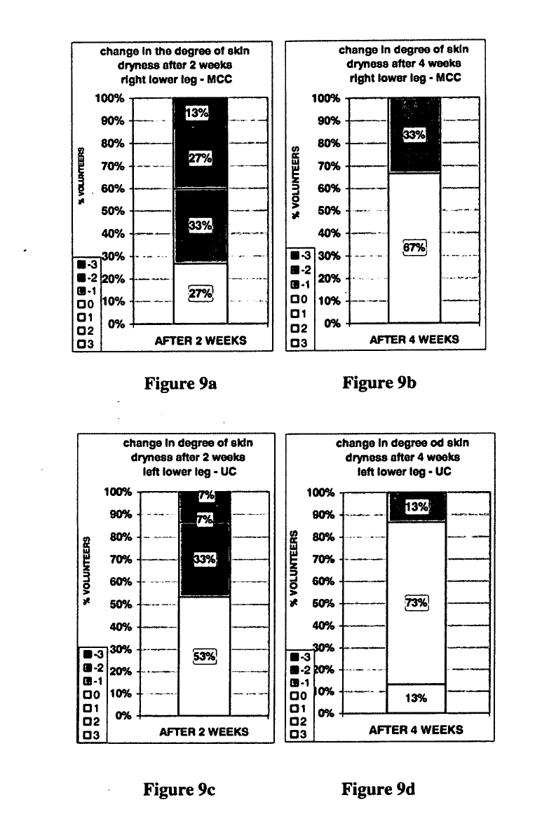

Table 13 above presents the investigators evaluations of the change in skin dryness on the lower legs of subjects treated with MCC (right lower leg) and UC (left lower leg), after the two-weeks treatment and two additional weeks thereafter (4 weeks). The change in the degree of skin roughness after two and four weeks is further presented in FIGS. 9a and 9b (for MCC) and FIGS. 9c and 9d (for UC).

The results presented above show that the composition of the present invention (Composition 4, MCC) was much more effective than the commercially available control composition (UC) in reducing skin dryness. While treatment with MCC reduced skin dryness in 74% of the treated forearms and 74% of the treated lower legs, treatment with UC reduced skin dryness in only 34% of the forearms and 47% of the lower legs. The differences between the two compositions were also statistically significant (p<0.05), both after the two-week treatment period (day 14) and on day 28.

Itchiness: No significant differences between the two treatments on the parameter of itchiness were observed. Both treatments, used daily, resulted in improvement (in most cases a total improvement) of itching.

Subjective Measurements:

The self-assessment of the participants (subjects) regarding skin dryness and itchiness are summarized in Table 14 below. The assessments are expressed as the effect of the treatment on skin dryness and itchiness (improved/no change/deterioration) during the two-weeks treatments (day 14) and two weeks thereafter (day 28).

As is shown in Table 14, according to the subjective measurements, MCC was evaluated as more effective than UC at reducing skin dryness and slightly more effective at reducing itchiness.

TABLE 14

| | | | Composition 4 (MCC) | | Control (UC) | |
|---|---|---|---|---|---|---|
| | | | Day 14 | Day 28 | Day 14 | Day 28 |
| | | Treated | No. (%) of | No. (%) of | No. (%) of | No. (%) of |
| Parameter | Change | Area | respondents | respondents | respondents | respondents |
| Skin | Improved | forearm | 11 (74) | 9 (60) | 5 (34) | 4 (27) |
| Dryness | | lower leg | 11 (74) | 5 (34) | 7 (47) | 2 (13) |
| | Same | forearm | 4 (27) | 6 (40) | 5 (33) | 8 (53) |
| | | lower leg | 4 (27) | 10 (67) | 8 (53) | 11 (73) |
| | Deteriorated | forearm | 0 (0) | 0 (0) | 5 (33) | 3 (20) |
| | | lower leg | 0 (0) | 0 (0) | 0 (0) | 2 (13) |
| Itchiness | Improved | Forearm | 9 (60) | 9 (60) | 7 (47) | 8 (53) |
| | | lower leg | 11 (74) | 9 (60) | 10 (67) | 9 (60) |
| | Same | forearm | 5 (33) | 5 (33) | 7 (47) | 6 (40) |
| | | lower leg | 3 (20) | 6 (40) | 4 (27) | 5 (33) |
| | Deteriorated | forearm | 1 (7) | 1 (7) | 1 (7) | 1 (7) |
| | | lower leg | 1 (7) | 0 (0) | 0 (0) | 1 (7) |

The subjects self-assessment obtained in the preference questionnaire described above are summarized in Table 15 below. The subjects have evaluated the questioned parameters on a scale of 1 to 5 and the results are expressed as means and standard deviations.

As is shown in Table 15, the composition of the present invention was evaluated by the participants as superior to the commercially available control composition in almost all the questioned parameters. For example, the Preference Questionnaire indicated that subjects significantly (p<0.05) preferred MCC over UC on all parameters related to the smell of the compositions. The Questionnaire also indicated a preference for MCC over UC with regard to its effect on skin texture.

TABLE 15

| | Composition 4 (MCC) | | Control (UC) | |
|---|---|---|---|---|
| Parameter | Day 0 | Day 14 | Day 0 | Day 14 |
| Skin roughness | 2.07 ± 0.70 | 2.00 ± 0.93 | 2.73 ± 1.33 | 2.47 ± 0.83 |
| Skin | | 3.67 ± 1.18 | | 3.33 ± 0.72 |
| smoothness | | | | |
| Skin texture | 4.47 ± 0.83 | 3.33 ± 1.35 | 3.33 ± 1.54 | 3.27 ± 0.96 |
| “in-package” | 3.60 ± 0.91 | | 2.33 ± 1.23 | |
| odor | | | | |
| Odor quality | | 3.13 ± 1.25 | | 1.79 ± 1.05 |
| Odor strength | 2.93 ± 1.28 | 2.60 ± 0.99 | 3.20 ± 1.42 | 3.21 ± 1.48 |
| Skin smell | 3.20 ± 1.15 | 3.33 ± 0.90 | 2.33 ± 1.11 | 2.47 ± 1.13 |
| Skin irritation | 1.00 ± 0.00 | 2.07 ± 1.39 | 1.40 ± 1.06 | 2.07 ± 1.53 |
| Stickiness | 2.00 ± 1.41 | 3.14 ± 1.41 | 2.07 ± 1.16 | 2.64 ± 1.45 |
| Remains of | 1.00 ± 0.00 | 1.64 ± 1.01 | 1.13 ± 0.52 | 2.14 ± 1.56 |
| white layer | | | | |
| Absorption | 4.07 ± 1/16 | 3.00 ± 1.18 | 4.53 ± 0.83 | 3.43 ± 1.16 |
| properties | | | | |
| Usage | 4.33 ± 1.18 | 3.64 ± 1.34 | 4.40 ± 0.91 | 3.14 ± 1.35 |
| comfortability | | | | |

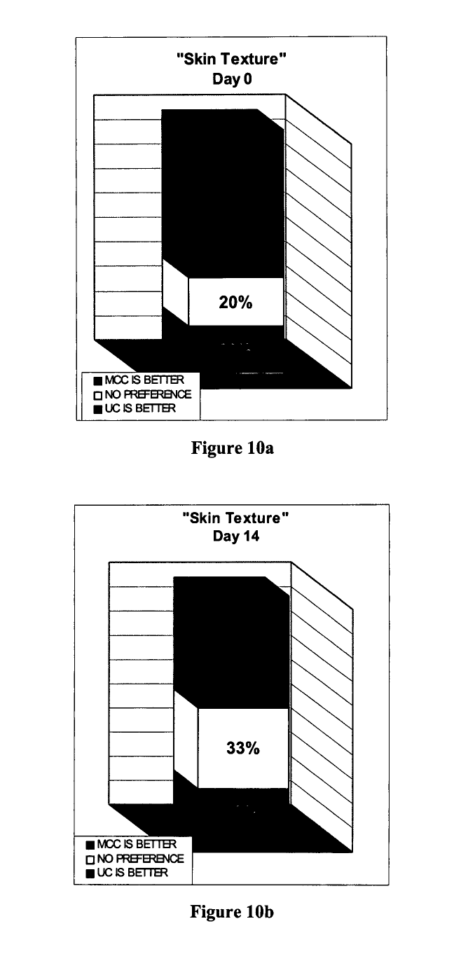

Detailed evaluations of the subjects regarding parameters related to odor and smell are presented in Tables 16-19 below and in FIGS. 10-13, as follows:

Table 16 presents the subjects evaluations regarding “skin texture”, in terms of the preferred composition (which resulted in nicer skin texture) at day 0 and day 14. FIGS. 10a and 10b further demonstrate the distribution of the subjects' preference in this regard at day 0 (FIG. 10a) and at day 14 (FIG. 10b).

TABLE 16

| Day 0 | Preferred | Day 14 |
|---|---|---|
| [No. of respondents (%)] | Composition | [No. of respondents (%)] |
| 9 (60) | MCC | 7 (47) |
| 3 (20) | No Preference | 5 (33) |
| 3 (20) | UC | 3 (20) |

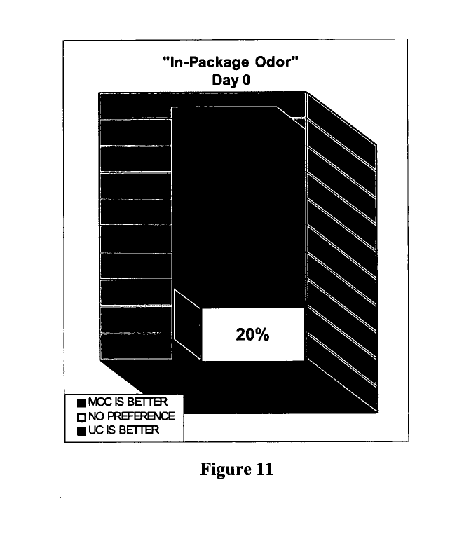

Table 17 presents the subjects evaluations regarding “in-package odor”, in terms of the preferred composition (having a better in-package odor) at day 0. FIG. 11 further demonstrates the distribution of the subjects' preference in this regard.

TABLE 17

| Day 0 | |
|---|---|
| [No. of respondents (%)] | Preferred Composition |
| 10 (67) | MCC |
| 3 (20) | No Preference |
| 2 (13) | UC |

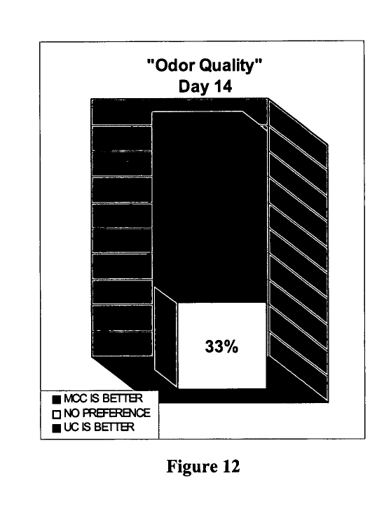

Table 18 presents the subjects evaluations regarding “odor quality”, in terms of the preferred composition (having a better odor) at day 14. FIG. 12 further demonstrates the distribution of the subjects' preference in this regard.

TABLE 18

| Day 14 | |
|---|---|
| [No. of respondents (%)] | Preferred Composition |
| 10 (67) | MCC |
| 5 (33) | No Preference |
| 0 (0) | UC |

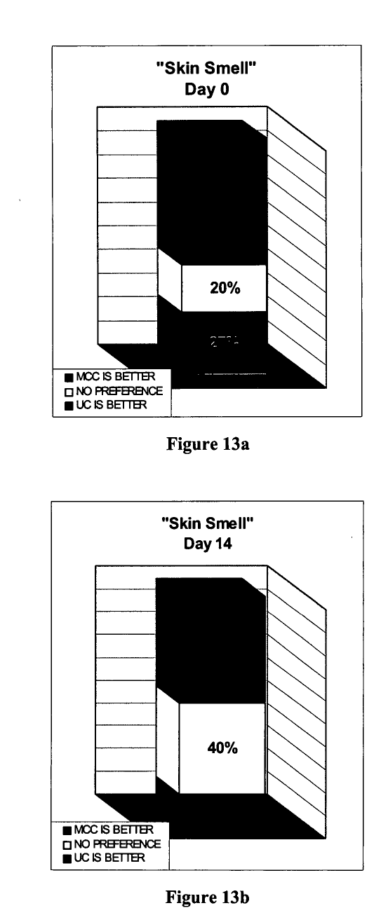

Table 19 presents the subjects evaluations regarding “skin smell”, in terms of the preferred composition (which resulted in better smell of the skin) at day 0 and day 14. FIGS. 13a and 13b further demonstrate the distribution of the subjects' preference in this regard at day 0 (FIG. 13a) and at day 14 (FIG. 13b).

TABLE 19

| Day 0 | Preferred | Day 14 |
|---|---|---|
| [No. of respondents (%)] | Composition | [No. of respondents (%)] |
| 8 (53) | MCC | 7 (47) |
| 3 (20) | No Preference | 6 (40) |
| 4 (27) | UC | 2 (13) |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A pharmaceutical, cosmetic or cosmeceutical composition for topical application comprising, as active ingredients, urea and ammonium lactate, and a pharmaceutically, cosmetically or cosmeceutically acceptable carrier, wherein the concentration of said urea is greater than 5 weight percentages of the composition and the concentration of said ammonium lactate is greater than 5 weight percentages of the composition, the composition being in a form selected from the group consisting of a soap, a lotion and an emulsion.

2. The pharmaceutical, cosmetic or cosmeceutical composition of claim 1, for use in the treatment of dermatitis.

3. The pharmaceutical, cosmetic or cosmeceutical composition of claim 1, wherein the concentration of said urea ranges between about 15 weight percentages and about 40 weight percentages of the composition.

4. The pharmaceutical, cosmetic or cosmeceutical composition of claim 3, wherein the concentration of said urea is about 20 weight percentages of the composition.

5. The pharmaceutical, cosmetic or cosmeceutical composition of claim 1, wherein the concentration of said ammonium lactate ranges between about 8 weight percentages and about 16 weight percentages of the composition.

6. The pharmaceutical, cosmetic or cosmeceutical composition of claim 5, wherein the concentration of said ammonium lactate is about 12 weight percentages of the composition.

7. The pharmaceutical, cosmetic or cosmeceutical composition of claim 1, further comprising at least one additional active ingredient, said at least one additional active ingredient being selected from the group consisting of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

8. The pharmaceutical, cosmetic or cosmeceutical composition of claim 1, further comprising at least one ingredient selected from the group consisting of a humectant, a conditioning agent, a pH adjusting agent, a chelating agent, a preservative, an emulsifier, an occlusive agent, an emollient, a thickener, a solubilizing agent, a penetration enhancer, an anti-irritant, a colorant, an antioxidant, a surfactant and an alcohol.

9. The pharmaceutical, cosmetic or cosmeceutical composition of claim 1, having a pH value that ranges between about 4 and about 7.

10. A method of treating dermatitis, the method comprising topically applying onto at least one biological surface of a subject in need thereof, a pharmaceutically, cosmetically or cosmeceutically effective amount of a pharmaceutical, cosmetic or cosmeceutical composition which comprises, as active ingredients, urea and ammonium lactate, and a pharmaceutically, cosmetically or cosmeceutically acceptable carrier, wherein the concentration of said urea is greater than 5 weight percentages of the composition and the concentration of said ammonium lactate is greater than 5 weight percentages of the composition, the composition being in a form selected from the group consisting of a soap, a lotion and an emulsion.

11. The method of claim 10, wherein said topically applying is performed between one and four times a day.

12. The method of claim 10, wherein said topically applying is for a time period that ranges between about 1 day and about 30 days.

13. The method of claim 10, wherein the concentration of said urea ranges between about 15 weight percentages and about 40 weight percentages of said composition.

14. The method of claim 13, wherein the concentration of said urea is about 20 weight percentages of said composition.

15. The method of claim 10, wherein the concentration of said ammonium lactate ranges between about 8 weight percentages and about 16 weight percentages of said composition.

16. The method of claim 15, wherein the concentration of said ammonium lactate is about 12 weight percentages of said composition.

17. The method of claim 10, wherein said composition further comprises at least one additional active ingredient, said at least one additional active ingredient being selected from the group consisting of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

18. The method of claim 10, wherein said composition further comprises at least one ingredient selected from the group consisting of a humectant, a conditioning agent, a pH adjusting agent, a chelating agent, a preservative, an emulsifier, an occlusive agent, an emollient, a thickener, a solubilizing agent, a penetration enhancer, an anti-irritant, a colorant, an antioxidant, a surfactant, and an alcohol.

19. The method of claim 10, wherein said composition has a pH value that ranges between about 4 and about 7.
